SEQUENCE LISTING (69 sequences)

SEQ ID NO: 1 - DNA, 1622 bp, Artificial Sequence
  intron 6
gtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccag

SEQ ID NO: 2 - DNA, 1488 bp, Artificial Sequence
  intron 7
gtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgccccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctggaagagggatgtaacattgccctgaggatggtgaagttggtatacatttataaagtacggaatggtgtcaatgaatgcaattctatgtatatggacttaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccatctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctctgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaag

SEQ ID NO: 3 - DNA, 1325 bp, Artificial Sequence
  intron 6 delta 1
gtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccag

SEQ ID NO: 4 - DNA, 1424 bp, Artificial Sequence
  intron 6 delta 2
gtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccag

SEQ ID NO: 5 - DNA, 1290 bp, Artificial Sequence
  intron 7 delta 3
gtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgcctggaagagggatgtaacattgccctgaggatggtgaagttggtatacatttataaagtacggaatggtgtcaatgaatgcaattctatgtatatggacttaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccatctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctctgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaag

SEQ ID NO: 6 - DNA, 1290 bp, Artificial Sequence
  intron 7 delta 4
gtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgccccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctggaagagggatgtaacattgccctgaggatggtgaagttggtatacatttataaagtacggaatggtgtcaatgaatgcaattctatgtatatggacttggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccatctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctctgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaag

SEQ ID NO: 7 - DNA, 1290 bp, Artificial Sequence
  intron 7 delta 5
gtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgccccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctggaagagggatgtaacattgccctgaggatggtgaagttggtatacatttataaagtacggaatggtgtcaatgaatgcaattctatgtatatggacttaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctggaacttttctggtttggttaacaaaaggtttcttgatttgtttcaagatttaaagccaaaggtgtgggttcatgacttaggtgtcattgcgtgtgggaaagatgaaattaaaaattgttccacaagggttaagtgtctggtggtaaagttgggagaaactggatggatgcacatcgcatggctggtggcgagcccatctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctctgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaag

SEQ ID NO: 8 - DNA, 1290 bp, Artificial Sequence
  intron 7 delta 6
gtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgccccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctggaagagggatgtaacattgccctgaggatggtgaagttggtatacatttataaagtacggaatggtgtcaatgaatgcaattctatgtatatggacttaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccactgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaag

SEQ ID NO: 9 - DNA, 4796 bp, Artificial Sequence
  squence complte sans 3′UTR
acttaagcttcgacgcaggaggcggggctgctcagtcctccaggcgtcggtactcagcggtgttggaacttcgttgcttgcttgcctgtgcgcgcgtgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccagcggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgccccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctggaagagggatgtaacattgccctgaggatggtgaagttggtatacatttataaagtacggaatggtgtcaatgaatgcaattctatgtatatggacttaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccatctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctctgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 10 - DNA, 8229 bp, Artificial Sequence
  sequence complte
acttaagcttcgacgcaggaggcggggctgctcagtcctccaggcgtcggtactcagcggtgttggaacttcgttgcttgcttgcctgtgcgcgcgtgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccagcggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgccccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctggaagagggatgtaacattgccctgaggatggtgaagttggtatacatttataaagtacggaatggtgtcaatgaatgcaattctatgtatatggacttaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccatctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctctgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaattagcctggctccccaggttctggaacagctttttgtcctgtacccagtgttaccctcgttattttgtaaccttccaattcctgatcacccaagggtttttttgtgtcggactatgtaattgtaactatacctctggttcccattaaaagtgaccattttagttaaattttgttcctcttcccccttttcactttcctggaagatcgatgtcccgatcaggaaggtagagagttttcctgttcagattaccctgcccagcaggaactggaatacagtgttcggggagaaggccaaatgatatccttgagagcagagattaaacttttctgtcatggggaaagttggtgtataaatgagaaatgaagaacatgggatgtcatgagtgttggcctaaatttgcccagctatggggaatttttcctttaccacatttatttgcatactggtcttagtttatttgcagcagtttatccctttttaagaactctttgatcttttggcccttttaatggtgaggctcaaacaaactacatttaaatggggcagtattcagatttgaccatggtggagagcgcttagccactctgggtctttcacaggaaggagagtaactgagtgctgcaggagtttgtggagtggagtcaggatctaggaggtgagtgactcccttcctagctgccctggtgaacagcgcttgggtagatacctgctataaggagactggtctggctgggttactttcacatcctgcctgtactcagagggcttgaggtcattgacattatgagattttaggcttgatccctttttgattggagggtggaaggccctcctaagggaatgataagtgataagagggggaaggggttgcagccaatgagttaaaaccttagagcagtgctcctcagcctcttaccatgtggttgtaaacttgcacgtacctgccaaccagttatttagcatgctttttattttagttacacagagcgtaacattaacccaagagcagaaaggttttatttacagggttttcgaacttggtttgtaagacagctgccatcacaagcatagcttacaaatgtgctggggacccctaattgggaagtgctttcctctcaaatttttattttttatttttagagacagagtcttgctctgtcatccaggctggagtgcagtggcgtgatctcggctcactgtagcctctgcctcctgagtttaagcgattctcctgcctcaggagaatcccagcttctgagtagctgagactacaggcgtgggccaccatgcccacctagtttttgcatttttagtggaggtgtggtttcactgtgttggccaggctggtctcttaactcctgacctcaggtgatccacctgccttggcctcccaaagtgctggaattacaggcatgagccgctgcatctggccatcctctcaaattttcaagtgttccacaagtatgttctctactgaagagttgctgcatccttgaatcttgggtgatttgaggcacagaaactatgactttattttttgagatggagttttgctcttgttgcccaggctggagtgcaatggcacgtttttcggcttaccgcaactgccgcctcctgggttcaagcgatagctgggattacaggcatgcgccaccatgcccagctaatttatttgtatttttagtagagacggggtttctccatgttggtcaggctggtctcgaactcccgacctcaggtgatgtgcacacctcagcctcccaataaaccatgacttttaagaggaatagcaggtttacttcccctgccagcattggggtgctctctaagcaacagtaggcggagagtggtctggcgtattaaaaacaaaggatcgtcaagtgggccttcccaggcattgctttgacttagtacatgtagaggatgtggcagttctctccgtccctgccactgctggtttctttgttaaatgtttagttgaaatggcctgatacgatatttgagtagttcactgttggtgctttgcctagcaggattctaatcttgctttggttgtggtcccctgatgccctcctgttaggagtggaggaggtcgaagctccttgtaagatatgattactgggaccattagtgtcaagttcctgtgtccttcaaatggcatatgtgattggccttgaccttaaaaggaaatagggtcccaggtgactgtttagtgggtaggtccagtttggggggatcttccaggagaatggatagagacacctagcagcagagagaacattggtgcctctcaagccaacctcccacctcagcctcccaagtagctgggactacaggtgcttcctcgctaccacacctgggtaattttttttttaattacttttttttttttaagaaacagggtttcactgggttgcccaggctggtcttgaactggcctcaagtgacctgcctgcctcagcctcccaaagtgctgggatcacaggtgtgacccactgcgtttggccagaatactctattcttactgaatgattgaaatctgtcttgaagcattaggtgtcccatttttgtgagttggaattgggacaggctaagtaggaagtgaggagggtggggagagctgtgctgtaggtctgtttgtcccttccttgatgtagccttcagttagccctttcagcttttttccccatcttgtgccgggccttcctgggtttcagtacttggatgtagggctgcagttatgtcagtggtgggtagattgaccaggaattaaggtctagggtccagcccatgtgagacttgactcactgatctacctttaggcatgtcttccttccagtctcatcctttttaaattttttttttttttttgagacggtctcactctcacccaggctggattgcagtggtgtgatctcgaccaactgcaacctctgcctcccacccgcaagctatctgcccacctcagcctctggagtagctgggacgggactacaggcacctgccaccatgactggctaatttttttttgtattttttgtggagatggggtcttgccatgttgctcaggctggtctggtctcaaaactgctctgggctcaagtgattgtcccactttggcctcccagagtgctgggattaaggtgtgagatactgtgtccggctatgaaaattttatttttaattaacttgtatatatttatggggtacaatgtcctatttctgtacatgtacacattgtggaatcaaatcaggctaatatatccatcacttcatatcattagcatgaatgagaacatacaaagccactcttagaaaattttgaaatttatgttatttcagcccttttatgctggaggttgcaaatgttttgtgaataatcagaccaaaaataaaaacaaaaaatgattgacttcagtcattcagtaagaa

SEQ ID NO: 11 - DNA, 7932 bp, Artificial Sequence
  sequence complete delta 1
acttaagcttcgacgcaggaggcggggctgctcagtcctccaggcgtcggtactcagcggtgttggaacttcgttgcttgcttgcctgtgcgcgcgtgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccagcggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgccccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctggaagagggatgtaacattgccctgaggatggtgaagttggtatacatttataaagtacggaatggtgtcaatgaatgcaattctatgtatatggacttaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccatctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctctgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaattagcctggctccccaggttctggaacagctttttgtcctgtacccagtgttaccctcgttattttgtaaccttccaattcctgatcacccaagggtttttttgtgtcggactatgtaattgtaactatacctctggttcccattaaaagtgaccattttagttaaattttgttcctcttcccccttttcactttcctggaagatcgatgtcccgatcaggaaggtagagagttttcctgttcagattaccctgcccagcaggaactggaatacagtgttcggggagaaggccaaatgatatccttgagagcagagattaaacttttctgtcatggggaaagttggtgtataaatgagaaatgaagaacatgggatgtcatgagtgttggcctaaatttgcccagctatggggaatttttcctttaccacatttatttgcatactggtcttagtttatttgcagcagtttatccctttttaagaactctttgatcttttggcccttttaatggtgaggctcaaacaaactacatttaaatggggcagtattcagatttgaccatggtggagagcgcttagccactctgggtctttcacaggaaggagagtaactgagtgctgcaggagtttgtggagtggagtcaggatctaggaggtgagtgactcccttcctagctgccctggtgaacagcgcttgggtagatacctgctataaggagactggtctggctgggttactttcacatcctgcctgtactcagagggcttgaggtcattgacattatgagattttaggcttgatccctttttgattggagggtggaaggccctcctaagggaatgataagtgataagagggggaaggggttgcagccaatgagttaaaaccttagagcagtgctcctcagcctcttaccatgtggttgtaaacttgcacgtacctgccaaccagttatttagcatgctttttattttagttacacagagcgtaacattaacccaagagcagaaaggttttatttacagggttttcgaacttggtttgtaagacagctgccatcacaagcatagcttacaaatgtgctggggacccctaattgggaagtgctttcctctcaaatttttattttttatttttagagacagagtcttgctctgtcatccaggctggagtgcagtggcgtgatctcggctcactgtagcctctgcctcctgagtttaagcgattctcctgcctcaggagaatcccagcttctgagtagctgagactacaggcgtgggccaccatgcccacctagtttttgcatttttagtggaggtgtggtttcactgtgttggccaggctggtctcttaactcctgacctcaggtgatccacctgccttggcctcccaaagtgctggaattacaggcatgagccgctgcatctggccatcctctcaaattttcaagtgttccacaagtatgttctctactgaagagttgctgcatccttgaatcttgggtgatttgaggcacagaaactatgactttattttttgagatggagttttgctcttgttgcccaggctggagtgcaatggcacgtttttcggcttaccgcaactgccgcctcctgggttcaagcgatagctgggattacaggcatgcgccaccatgcccagctaatttatttgtatttttagtagagacggggtttctccatgttggtcaggctggtctcgaactcccgacctcaggtgatgtgcacacctcagcctcccaataaaccatgacttttaagaggaatagcaggtttacttcccctgccagcattggggtgctctctaagcaacagtaggcggagagtggtctggcgtattaaaaacaaaggatcgtcaagtgggccttcccaggcattgctttgacttagtacatgtagaggatgtggcagttctctccgtccctgccactgctggtttctttgttaaatgtttagttgaaatggcctgatacgatatttgagtagttcactgttggtgctttgcctagcaggattctaatcttgctttggttgtggtcccctgatgccctcctgttaggagtggaggaggtcgaagctccttgtaagatatgattactgggaccattagtgtcaagttcctgtgtccttcaaatggcatatgtgattggccttgaccttaaaaggaaatagggtcccaggtgactgtttagtgggtaggtccagtttggggggatcttccaggagaatggatagagacacctagcagcagagagaacattggtgcctctcaagccaacctcccacctcagcctcccaagtagctgggactacaggtgcttcctcgctaccacacctgggtaattttttttttaattacttttttttttttaagaaacagggtttcactgggttgcccaggctggtcttgaactggcctcaagtgacctgcctgcctcagcctcccaaagtgctgggatcacaggtgtgacccactgcgtttggccagaatactctattcttactgaatgattgaaatctgtcttgaagcattaggtgtcccatttttgtgagttggaattgggacaggctaagtaggaagtgaggagggtggggagagctgtgctgtaggtctgtttgtcccttccttgatgtagccttcagttagccctttcagcttttttccccatcttgtgccgggccttcctgggtttcagtacttggatgtagggctgcagttatgtcagtggtgggtagattgaccaggaattaaggtctagggtccagcccatgtgagacttgactcactgatctacctttaggcatgtcttccttccagtctcatcctttttaaattttttttttttttttgagacggtctcactctcacccaggctggattgcagtggtgtgatctcgaccaactgcaacctctgcctcccacccgcaagctatctgcccacctcagcctctggagtagctgggacgggactacaggcacctgccaccatgactggctaatttttttttgtattttttgtggagatggggtcttgccatgttgctcaggctggtctggtctcaaaactgctctgggctcaagtgattgtcccactttggcctcccagagtgctgggattaaggtgtgagatactgtgtccggctatgaaaattttatttttaattaacttgtatatatttatggggtacaatgtcctatttctgtacatgtacacattgtggaatcaaatcaggctaatatatccatcacttcatatcattagcatgaatgagaacatacaaagccactcttagaaaattttgaaatttatgttatttcagcccttttatgctggaggttgcaaatgttttgtgaataatcagaccaaaaataaaaacaaaaaatgattgacttcagtcattcagtaagaa

SEQ ID NO: 12 - DNA, 8031 bp, Artificial Sequence
  sequence delta 2
acttaagcttcgacgcaggaggcggggctgctcagtcctccaggcgtcggtactcagcggtgttggaacttcgttgcttgcttgcctgtgcgcgcgtgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccagcggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgccccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctggaagagggatgtaacattgccctgaggatggtgaagttggtatacatttataaagtacggaatggtgtcaatgaatgcaattctatgtatatggacttaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccatctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctctgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaattagcctggctccccaggttctggaacagctttttgtcctgtacccagtgttaccctcgttattttgtaaccttccaattcctgatcacccaagggtttttttgtgtcggactatgtaattgtaactatacctctggttcccattaaaagtgaccattttagttaaattttgttcctcttcccccttttcactttcctggaagatcgatgtcccgatcaggaaggtagagagttttcctgttcagattaccctgcccagcaggaactggaatacagtgttcggggagaaggccaaatgatatccttgagagcagagattaaacttttctgtcatggggaaagttggtgtataaatgagaaatgaagaacatgggatgtcatgagtgttggcctaaatttgcccagctatggggaatttttcctttaccacatttatttgcatactggtcttagtttatttgcagcagtttatccctttttaagaactctttgatcttttggcccttttaatggtgaggctcaaacaaactacatttaaatggggcagtattcagatttgaccatggtggagagcgcttagccactctgggtctttcacaggaaggagagtaactgagtgctgcaggagtttgtggagtggagtcaggatctaggaggtgagtgactcccttcctagctgccctggtgaacagcgcttgggtagatacctgctataaggagactggtctggctgggttactttcacatcctgcctgtactcagagggcttgaggtcattgacattatgagattttaggcttgatccctttttgattggagggtggaaggccctcctaagggaatgataagtgataagagggggaaggggttgcagccaatgagttaaaaccttagagcagtgctcctcagcctcttaccatgtggttgtaaacttgcacgtacctgccaaccagttatttagcatgctttttattttagttacacagagcgtaacattaacccaagagcagaaaggttttatttacagggttttcgaacttggtttgtaagacagctgccatcacaagcatagcttacaaatgtgctggggacccctaattgggaagtgctttcctctcaaatttttattttttatttttagagacagagtcttgctctgtcatccaggctggagtgcagtggcgtgatctcggctcactgtagcctctgcctcctgagtttaagcgattctcctgcctcaggagaatcccagcttctgagtagctgagactacaggcgtgggccaccatgcccacctagtttttgcatttttagtggaggtgtggtttcactgtgttggccaggctggtctcttaactcctgacctcaggtgatccacctgccttggcctcccaaagtgctggaattacaggcatgagccgctgcatctggccatcctctcaaattttcaagtgttccacaagtatgttctctactgaagagttgctgcatccttgaatcttgggtgatttgaggcacagaaactatgactttattttttgagatggagttttgctcttgttgcccaggctggagtgcaatggcacgtttttcggcttaccgcaactgccgcctcctgggttcaagcgatagctgggattacaggcatgcgccaccatgcccagctaatttatttgtatttttagtagagacggggtttctccatgttggtcaggctggtctcgaactcccgacctcaggtgatgtgcacacctcagcctcccaataaaccatgacttttaagaggaatagcaggtttacttcccctgccagcattggggtgctctctaagcaacagtaggcggagagtggtctggcgtattaaaaacaaaggatcgtcaagtgggccttcccaggcattgctttgacttagtacatgtagaggatgtggcagttctctccgtccctgccactgctggtttctttgttaaatgtttagttgaaatggcctgatacgatatttgagtagttcactgttggtgctttgcctagcaggattctaatcttgctttggttgtggtcccctgatgccctcctgttaggagtggaggaggtcgaagctccttgtaagatatgattactgggaccattagtgtcaagttcctgtgtccttcaaatggcatatgtgattggccttgaccttaaaaggaaatagggtcccaggtgactgtttagtgggtaggtccagtttggggggatcttccaggagaatggatagagacacctagcagcagagagaacattggtgcctctcaagccaacctcccacctcagcctcccaagtagctgggactacaggtgcttcctcgctaccacacctgggtaattttttttttaattacttttttttttttaagaaacagggtttcactgggttgcccaggctggtcttgaactggcctcaagtgacctgcctgcctcagcctcccaaagtgctgggatcacaggtgtgacccactgcgtttggccagaatactctattcttactgaatgattgaaatctgtcttgaagcattaggtgtcccatttttgtgagttggaattgggacaggctaagtaggaagtgaggagggtggggagagctgtgctgtaggtctgtttgtcccttccttgatgtagccttcagttagccctttcagcttttttccccatcttgtgccgggccttcctgggtttcagtacttggatgtagggctgcagttatgtcagtggtgggtagattgaccaggaattaaggtctagggtccagcccatgtgagacttgactcactgatctacctttaggcatgtcttccttccagtctcatcctttttaaattttttttttttttttgagacggtctcactctcacccaggctggattgcagtggtgtgatctcgaccaactgcaacctctgcctcccacccgcaagctatctgcccacctcagcctctggagtagctgggacgggactacaggcacctgccaccatgactggctaatttttttttgtattttttgtggagatggggtcttgccatgttgctcaggctggtctggtctcaaaactgctctgggctcaagtgattgtcccactttggcctcccagagtgctgggattaaggtgtgagatactgtgtccggctatgaaaattttatttttaattaacttgtatatatttatggggtacaatgtcctatttctgtacatgtacacattgtggaatcaaatcaggctaatatatccatcacttcatatcattagcatgaatgagaacatacaaagccactcttagaaaattttgaaatttatgttatttcagcccttttatgctggaggttgcaaatgttttgtgaataatcagaccaaaaataaaaacaaaaaatgattgacttcagtcattcagtaagaa

SEQ ID NO: 13 - DNA, 8031 bp, Artificial Sequence
  sequence delta 3
acttaagcttcgacgcaggaggcggggctgctcagtcctccaggcgtcggtactcagcggtgttggaacttcgttgcttgcttgcctgtgcgcgcgtgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccagcggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgcctggaagagggatgtaacattgccctgaggatggtgaagttggtatacatttataaagtacggaatggtgtcaatgaatgcaattctatgtatatggacttaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccatctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctctgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaattagcctggctccccaggttctggaacagctttttgtcctgtacccagtgttaccctcgttattttgtaaccttccaattcctgatcacccaagggtttttttgtgtcggactatgtaattgtaactatacctctggttcccattaaaagtgaccattttagttaaattttgttcctcttcccccttttcactttcctggaagatcgatgtcccgatcaggaaggtagagagttttcctgttcagattaccctgcccagcaggaactggaatacagtgttcggggagaaggccaaatgatatccttgagagcagagattaaacttttctgtcatggggaaagttggtgtataaatgagaaatgaagaacatgggatgtcatgagtgttggcctaaatttgcccagctatggggaatttttcctttaccacatttatttgcatactggtcttagtttatttgcagcagtttatccctttttaagaactctttgatcttttggcccttttaatggtgaggctcaaacaaactacatttaaatggggcagtattcagatttgaccatggtggagagcgcttagccactctgggtctttcacaggaaggagagtaactgagtgctgcaggagtttgtggagtggagtcaggatctaggaggtgagtgactcccttcctagctgccctggtgaacagcgcttgggtagatacctgctataaggagactggtctggctgggttactttcacatcctgcctgtactcagagggcttgaggtcattgacattatgagattttaggcttgatccctttttgattggagggtggaaggccctcctaagggaatgataagtgataagagggggaaggggttgcagccaatgagttaaaaccttagagcagtgctcctcagcctcttaccatgtggttgtaaacttgcacgtacctgccaaccagttatttagcatgctttttattttagttacacagagcgtaacattaacccaagagcagaaaggttttatttacagggttttcgaacttggtttgtaagacagctgccatcacaagcatagcttacaaatgtgctggggacccctaattgggaagtgctttcctctcaaatttttattttttatttttagagacagagtcttgctctgtcatccaggctggagtgcagtggcgtgatctcggctcactgtagcctctgcctcctgagtttaagcgattctcctgcctcaggagaatcccagcttctgagtagctgagactacaggcgtgggccaccatgcccacctagtttttgcatttttagtggaggtgtggtttcactgtgttggccaggctggtctcttaactcctgacctcaggtgatccacctgccttggcctcccaaagtgctggaattacaggcatgagccgctgcatctggccatcctctcaaattttcaagtgttccacaagtatgttctctactgaagagttgctgcatccttgaatcttgggtgatttgaggcacagaaactatgactttattttttgagatggagttttgctcttgttgcccaggctggagtgcaatggcacgtttttcggcttaccgcaactgccgcctcctgggttcaagcgatagctgggattacaggcatgcgccaccatgcccagctaatttatttgtatttttagtagagacggggtttctccatgttggtcaggctggtctcgaactcccgacctcaggtgatgtgcacacctcagcctcccaataaaccatgacttttaagaggaatagcaggtttacttcccctgccagcattggggtgctctctaagcaacagtaggcggagagtggtctggcgtattaaaaacaaaggatcgtcaagtgggccttcccaggcattgctttgacttagtacatgtagaggatgtggcagttctctccgtccctgccactgctggtttctttgttaaatgtttagttgaaatggcctgatacgatatttgagtagttcactgttggtgctttgcctagcaggattctaatcttgctttggttgtggtcccctgatgccctcctgttaggagtggaggaggtcgaagctccttgtaagatatgattactgggaccattagtgtcaagttcctgtgtccttcaaatggcatatgtgattggccttgaccttaaaaggaaatagggtcccaggtgactgtttagtgggtaggtccagtttggggggatcttccaggagaatggatagagacacctagcagcagagagaacattggtgcctctcaagccaacctcccacctcagcctcccaagtagctgggactacaggtgcttcctcgctaccacacctgggtaattttttttttaattacttttttttttttaagaaacagggtttcactgggttgcccaggctggtcttgaactggcctcaagtgacctgcctgcctcagcctcccaaagtgctgggatcacaggtgtgacccactgcgtttggccagaatactctattcttactgaatgattgaaatctgtcttgaagcattaggtgtcccatttttgtgagttggaattgggacaggctaagtaggaagtgaggagggtggggagagctgtgctgtaggtctgtttgtcccttccttgatgtagccttcagttagccctttcagcttttttccccatcttgtgccgggccttcctgggtttcagtacttggatgtagggctgcagttatgtcagtggtgggtagattgaccaggaattaaggtctagggtccagcccatgtgagacttgactcactgatctacctttaggcatgtcttccttccagtctcatcctttttaaattttttttttttttttgagacggtctcactctcacccaggctggattgcagtggtgtgatctcgaccaactgcaacctctgcctcccacccgcaagctatctgcccacctcagcctctggagtagctgggacgggactacaggcacctgccaccatgactggctaatttttttttgtattttttgtggagatggggtcttgccatgttgctcaggctggtctggtctcaaaactgctctgggctcaagtgattgtcccactttggcctcccagagtgctgggattaaggtgtgagatactgtgtccggctatgaaaattttatttttaattaacttgtatatatttatggggtacaatgtcctatttctgtacatgtacacattgtggaatcaaatcaggctaatatatccatcacttcatatcattagcatgaatgagaacatacaaagccactcttagaaaattttgaaatttatgttatttcagcccttttatgctggaggttgcaaatgttttgtgaataatcagaccaaaaataaaaacaaaaaatgattgacttcagtcattcagtaagaa

SEQ ID NO: 14 - DNA, 8031 bp, Artificial Sequence
  sequence delta 4
acttaagcttcgacgcaggaggcggggctgctcagtcctccaggcgtcggtactcagcggtgttggaacttcgttgcttgcttgcctgtgcgcgcgtgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccagcggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgccccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctggaagagggatgtaacattgccctgaggatggtgaagttggtatacatttataaagtacggaatggtgtcaatgaatgcaattctatgtatatggacttggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccatctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctctgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaattagcctggctccccaggttctggaacagctttttgtcctgtacccagtgttaccctcgttattttgtaaccttccaattcctgatcacccaagggtttttttgtgtcggactatgtaattgtaactatacctctggttcccattaaaagtgaccattttagttaaattttgttcctcttcccccttttcactttcctggaagatcgatgtcccgatcaggaaggtagagagttttcctgttcagattaccctgcccagcaggaactggaatacagtgttcggggagaaggccaaatgatatccttgagagcagagattaaacttttctgtcatggggaaagttggtgtataaatgagaaatgaagaacatgggatgtcatgagtgttggcctaaatttgcccagctatggggaatttttcctttaccacatttatttgcatactggtcttagtttatttgcagcagtttatccctttttaagaactctttgatcttttggcccttttaatggtgaggctcaaacaaactacatttaaatggggcagtattcagatttgaccatggtggagagcgcttagccactctgggtctttcacaggaaggagagtaactgagtgctgcaggagtttgtggagtggagtcaggatctaggaggtgagtgactcccttcctagctgccctggtgaacagcgcttgggtagatacctgctataaggagactggtctggctgggttactttcacatcctgcctgtactcagagggcttgaggtcattgacattatgagattttaggcttgatccctttttgattggagggtggaaggccctcctaagggaatgataagtgataagagggggaaggggttgcagccaatgagttaaaaccttagagcagtgctcctcagcctcttaccatgtggttgtaaacttgcacgtacctgccaaccagttatttagcatgctttttattttagttacacagagcgtaacattaacccaagagcagaaaggttttatttacagggttttcgaacttggtttgtaagacagctgccatcacaagcatagcttacaaatgtgctggggacccctaattgggaagtgctttcctctcaaatttttattttttatttttagagacagagtcttgctctgtcatccaggctggagtgcagtggcgtgatctcggctcactgtagcctctgcctcctgagtttaagcgattctcctgcctcaggagaatcccagcttctgagtagctgagactacaggcgtgggccaccatgcccacctagtttttgcatttttagtggaggtgtggtttcactgtgttggccaggctggtctcttaactcctgacctcaggtgatccacctgccttggcctcccaaagtgctggaattacaggcatgagccgctgcatctggccatcctctcaaattttcaagtgttccacaagtatgttctctactgaagagttgctgcatccttgaatcttgggtgatttgaggcacagaaactatgactttattttttgagatggagttttgctcttgttgcccaggctggagtgcaatggcacgtttttcggcttaccgcaactgccgcctcctgggttcaagcgatagctgggattacaggcatgcgccaccatgcccagctaatttatttgtatttttagtagagacggggtttctccatgttggtcaggctggtctcgaactcccgacctcaggtgatgtgcacacctcagcctcccaataaaccatgacttttaagaggaatagcaggtttacttcccctgccagcattggggtgctctctaagcaacagtaggcggagagtggtctggcgtattaaaaacaaaggatcgtcaagtgggccttcccaggcattgctttgacttagtacatgtagaggatgtggcagttctctccgtccctgccactgctggtttctttgttaaatgtttagttgaaatggcctgatacgatatttgagtagttcactgttggtgctttgcctagcaggattctaatcttgctttggttgtggtcccctgatgccctcctgttaggagtggaggaggtcgaagctccttgtaagatatgattactgggaccattagtgtcaagttcctgtgtccttcaaatggcatatgtgattggccttgaccttaaaaggaaatagggtcccaggtgactgtttagtgggtaggtccagtttggggggatcttccaggagaatggatagagacacctagcagcagagagaacattggtgcctctcaagccaacctcccacctcagcctcccaagtagctgggactacaggtgcttcctcgctaccacacctgggtaattttttttttaattacttttttttttttaagaaacagggtttcactgggttgcccaggctggtcttgaactggcctcaagtgacctgcctgcctcagcctcccaaagtgctgggatcacaggtgtgacccactgcgtttggccagaatactctattcttactgaatgattgaaatctgtcttgaagcattaggtgtcccatttttgtgagttggaattgggacaggctaagtaggaagtgaggagggtggggagagctgtgctgtaggtctgtttgtcccttccttgatgtagccttcagttagccctttcagcttttttccccatcttgtgccgggccttcctgggtttcagtacttggatgtagggctgcagttatgtcagtggtgggtagattgaccaggaattaaggtctagggtccagcccatgtgagacttgactcactgatctacctttaggcatgtcttccttccagtctcatcctttttaaattttttttttttttttgagacggtctcactctcacccaggctggattgcagtggtgtgatctcgaccaactgcaacctctgcctcccacccgcaagctatctgcccacctcagcctctggagtagctgggacgggactacaggcacctgccaccatgactggctaatttttttttgtattttttgtggagatggggtcttgccatgttgctcaggctggtctggtctcaaaactgctctgggctcaagtgattgtcccactttggcctcccagagtgctgggattaaggtgtgagatactgtgtccggctatgaaaattttatttttaattaacttgtatatatttatggggtacaatgtcctatttctgtacatgtacacattgtggaatcaaatcaggctaatatatccatcacttcatatcattagcatgaatgagaacatacaaagccactcttagaaaattttgaaatttatgttatttcagcccttttatgctggaggttgcaaatgttttgtgaataatcagaccaaaaataaaaacaaaaaatgattgacttcagtcattcagtaagaa

SEQ ID NO: 15 - DNA, 8031 bp, Artificial Sequence
  squence delta 5
acttaagcttcgacgcaggaggcggggctgctcagtcctccaggcgtcggtactcagcggtgttggaacttcgttgcttgcttgcctgtgcgcgcgtgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccagcggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgccccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctggaagagggatgtaacattgccctgaggatggtgaagttggtatacatttataaagtacggaatggtgtcaatgaatgcaattctatgtatatggacttaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctggaacttttctggtttggttaacaaaaggtttcttgatttgtttcaagatttaaagccaaaggtgtgggttcatgacttaggtgtcattgcgtgtgggaaagatgaaattaaaaattgttccacaagggttaagtgtctggtggtaaagttgggagaaactggatggatgcacatcgcatggctggtggcgagcccatctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctctgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaattagcctggctccccaggttctggaacagctttttgtcctgtacccagtgttaccctcgttattttgtaaccttccaattcctgatcacccaagggtttttttgtgtcggactatgtaattgtaactatacctctggttcccattaaaagtgaccattttagttaaattttgttcctcttcccccttttcactttcctggaagatcgatgtcccgatcaggaaggtagagagttttcctgttcagattaccctgcccagcaggaactggaatacagtgttcggggagaaggccaaatgatatccttgagagcagagattaaacttttctgtcatggggaaagttggtgtataaatgagaaatgaagaacatgggatgtcatgagtgttggcctaaatttgcccagctatggggaatttttcctttaccacatttatttgcatactggtcttagtttatttgcagcagtttatccctttttaagaactctttgatcttttggcccttttaatggtgaggctcaaacaaactacatttaaatggggcagtattcagatttgaccatggtggagagcgcttagccactctgggtctttcacaggaaggagagtaactgagtgctgcaggagtttgtggagtggagtcaggatctaggaggtgagtgactcccttcctagctgccctggtgaacagcgcttgggtagatacctgctataaggagactggtctggctgggttactttcacatcctgcctgtactcagagggcttgaggtcattgacattatgagattttaggcttgatccctttttgattggagggtggaaggccctcctaagggaatgataagtgataagagggggaaggggttgcagccaatgagttaaaaccttagagcagtgctcctcagcctcttaccatgtggttgtaaacttgcacgtacctgccaaccagttatttagcatgctttttattttagttacacagagcgtaacattaacccaagagcagaaaggttttatttacagggttttcgaacttggtttgtaagacagctgccatcacaagcatagcttacaaatgtgctggggacccctaattgggaagtgctttcctctcaaatttttattttttatttttagagacagagtcttgctctgtcatccaggctggagtgcagtggcgtgatctcggctcactgtagcctctgcctcctgagtttaagcgattctcctgcctcaggagaatcccagcttctgagtagctgagactacaggcgtgggccaccatgcccacctagtttttgcatttttagtggaggtgtggtttcactgtgttggccaggctggtctcttaactcctgacctcaggtgatccacctgccttggcctcccaaagtgctggaattacaggcatgagccgctgcatctggccatcctctcaaattttcaagtgttccacaagtatgttctctactgaagagttgctgcatccttgaatcttgggtgatttgaggcacagaaactatgactttattttttgagatggagttttgctcttgttgcccaggctggagtgcaatggcacgtttttcggcttaccgcaactgccgcctcctgggttcaagcgatagctgggattacaggcatgcgccaccatgcccagctaatttatttgtatttttagtagagacggggtttctccatgttggtcaggctggtctcgaactcccgacctcaggtgatgtgcacacctcagcctcccaataaaccatgacttttaagaggaatagcaggtttacttcccctgccagcattggggtgctctctaagcaacagtaggcggagagtggtctggcgtattaaaaacaaaggatcgtcaagtgggccttcccaggcattgctttgacttagtacatgtagaggatgtggcagttctctccgtccctgccactgctggtttctttgttaaatgtttagttgaaatggcctgatacgatatttgagtagttcactgttggtgctttgcctagcaggattctaatcttgctttggttgtggtcccctgatgccctcctgttaggagtggaggaggtcgaagctccttgtaagatatgattactgggaccattagtgtcaagttcctgtgtccttcaaatggcatatgtgattggccttgaccttaaaaggaaatagggtcccaggtgactgtttagtgggtaggtccagtttggggggatcttccaggagaatggatagagacacctagcagcagagagaacattggtgcctctcaagccaacctcccacctcagcctcccaagtagctgggactacaggtgcttcctcgctaccacacctgggtaattttttttttaattacttttttttttttaagaaacagggtttcactgggttgcccaggctggtcttgaactggcctcaagtgacctgcctgcctcagcctcccaaagtgctgggatcacaggtgtgacccactgcgtttggccagaatactctattcttactgaatgattgaaatctgtcttgaagcattaggtgtcccatttttgtgagttggaattgggacaggctaagtaggaagtgaggagggtggggagagctgtgctgtaggtctgtttgtcccttccttgatgtagccttcagttagccctttcagcttttttccccatcttgtgccgggccttcctgggtttcagtacttggatgtagggctgcagttatgtcagtggtgggtagattgaccaggaattaaggtctagggtccagcccatgtgagacttgactcactgatctacctttaggcatgtcttccttccagtctcatcctttttaaattttttttttttttttgagacggtctcactctcacccaggctggattgcagtggtgtgatctcgaccaactgcaacctctgcctcccacccgcaagctatctgcccacctcagcctctggagtagctgggacgggactacaggcacctgccaccatgactggctaatttttttttgtattttttgtggagatggggtcttgccatgttgctcaggctggtctggtctcaaaactgctctgggctcaagtgattgtcccactttggcctcccagagtgctgggattaaggtgtgagatactgtgtccggctatgaaaattttatttttaattaacttgtatatatttatggggtacaatgtcctatttctgtacatgtacacattgtggaatcaaatcaggctaatatatccatcacttcatatcattagcatgaatgagaacatacaaagccactcttagaaaattttgaaatttatgttatttcagcccttttatgctggaggttgcaaatgttttgtgaataatcagaccaaaaataaaaacaaaaaatgattgacttcagtcattcagtaagaa

SEQ ID NO: 16 - DNA, 8031 bp, Artificial Sequence
  sequence delta 6
acttaagcttcgacgcaggaggcggggctgctcagtcctccaggcgtcggtactcagcggtgttggaacttcgttgcttgcttgcctgtgcgcgcgtgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccagcggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgccccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctggaagagggatgtaacattgccctgaggatggtgaagttggtatacatttataaagtacggaatggtgtcaatgaatgcaattctatgtatatggacttaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccactgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaattagcctggctccccaggttctggaacagctttttgtcctgtacccagtgttaccctcgttattttgtaaccttccaattcctgatcacccaagggtttttttgtgtcggactatgtaattgtaactatacctctggttcccattaaaagtgaccattttagttaaattttgttcctcttcccccttttcactttcctggaagatcgatgtcccgatcaggaaggtagagagttttcctgttcagattaccctgcccagcaggaactggaatacagtgttcggggagaaggccaaatgatatccttgagagcagagattaaacttttctgtcatggggaaagttggtgtataaatgagaaatgaagaacatgggatgtcatgagtgttggcctaaatttgcccagctatggggaatttttcctttaccacatttatttgcatactggtcttagtttatttgcagcagtttatccctttttaagaactctttgatcttttggcccttttaatggtgaggctcaaacaaactacatttaaatggggcagtattcagatttgaccatggtggagagcgcttagccactctgggtctttcacaggaaggagagtaactgagtgctgcaggagtttgtggagtggagtcaggatctaggaggtgagtgactcccttcctagctgccctggtgaacagcgcttgggtagatacctgctataaggagactggtctggctgggttactttcacatcctgcctgtactcagagggcttgaggtcattgacattatgagattttaggcttgatccctttttgattggagggtggaaggccctcctaagggaatgataagtgataagagggggaaggggttgcagccaatgagttaaaaccttagagcagtgctcctcagcctcttaccatgtggttgtaaacttgcacgtacctgccaaccagttatttagcatgctttttattttagttacacagagcgtaacattaacccaagagcagaaaggttttatttacagggttttcgaacttggtttgtaagacagctgccatcacaagcatagcttacaaatgtgctggggacccctaattgggaagtgctttcctctcaaatttttattttttatttttagagacagagtcttgctctgtcatccaggctggagtgcagtggcgtgatctcggctcactgtagcctctgcctcctgagtttaagcgattctcctgcctcaggagaatcccagcttctgagtagctgagactacaggcgtgggccaccatgcccacctagtttttgcatttttagtggaggtgtggtttcactgtgttggccaggctggtctcttaactcctgacctcaggtgatccacctgccttggcctcccaaagtgctggaattacaggcatgagccgctgcatctggccatcctctcaaattttcaagtgttccacaagtatgttctctactgaagagttgctgcatccttgaatcttgggtgatttgaggcacagaaactatgactttattttttgagatggagttttgctcttgttgcccaggctggagtgcaatggcacgtttttcggcttaccgcaactgccgcctcctgggttcaagcgatagctgggattacaggcatgcgccaccatgcccagctaatttatttgtatttttagtagagacggggtttctccatgttggtcaggctggtctcgaactcccgacctcaggtgatgtgcacacctcagcctcccaataaaccatgacttttaagaggaatagcaggtttacttcccctgccagcattggggtgctctctaagcaacagtaggcggagagtggtctggcgtattaaaaacaaaggatcgtcaagtgggccttcccaggcattgctttgacttagtacatgtagaggatgtggcagttctctccgtccctgccactgctggtttctttgttaaatgtttagttgaaatggcctgatacgatatttgagtagttcactgttggtgctttgcctagcaggattctaatcttgctttggttgtggtcccctgatgccctcctgttaggagtggaggaggtcgaagctccttgtaagatatgattactgggaccattagtgtcaagttcctgtgtccttcaaatggcatatgtgattggccttgaccttaaaaggaaatagggtcccaggtgactgtttagtgggtaggtccagtttggggggatcttccaggagaatggatagagacacctagcagcagagagaacattggtgcctctcaagccaacctcccacctcagcctcccaagtagctgggactacaggtgcttcctcgctaccacacctgggtaattttttttttaattacttttttttttttaagaaacagggtttcactgggttgcccaggctggtcttgaactggcctcaagtgacctgcctgcctcagcctcccaaagtgctgggatcacaggtgtgacccactgcgtttggccagaatactctattcttactgaatgattgaaatctgtcttgaagcattaggtgtcccatttttgtgagttggaattgggacaggctaagtaggaagtgaggagggtggggagagctgtgctgtaggtctgtttgtcccttccttgatgtagccttcagttagccctttcagcttttttccccatcttgtgccgggccttcctgggtttcagtacttggatgtagggctgcagttatgtcagtggtgggtagattgaccaggaattaaggtctagggtccagcccatgtgagacttgactcactgatctacctttaggcatgtcttccttccagtctcatcctttttaaattttttttttttttttgagacggtctcactctcacccaggctggattgcagtggtgtgatctcgaccaactgcaacctctgcctcccacccgcaagctatctgcccacctcagcctctggagtagctgggacgggactacaggcacctgccaccatgactggctaatttttttttgtattttttgtggagatggggtcttgccatgttgctcaggctggtctggtctcaaaactgctctgggctcaagtgattgtcccactttggcctcccagagtgctgggattaaggtgtgagatactgtgtccggctatgaaaattttatttttaattaacttgtatatatttatggggtacaatgtcctatttctgtacatgtacacattgtggaatcaaatcaggctaatatatccatcacttcatatcattagcatgaatgagaacatacaaagccactcttagaaaattttgaaatttatgttatttcagcccttttatgctggaggttgcaaatgttttgtgaataatcagaccaaaaataaaaacaaaaaatgattgacttcagtcattcagtaagaa

SEQ ID NO: 17 - DNA, 7140 bp, Artificial Sequence
  sequence delta 1-6
acttaagcttcgacgcaggaggcggggctgctcagtcctccaggcgtcggtactcagcggtgttggaacttcgttgcttgcttgcctgtgcgcgcgtgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccagcggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgcctggaagagggatgtaacattgccctgaggatggtgaagttggtatacatttataaagtacggaatggtgtcaatgaatgcaattctatgtatatggacttggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccactgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaattagcctggctccccaggttctggaacagctttttgtcctgtacccagtgttaccctcgttattttgtaaccttccaattcctgatcacccaagggtttttttgtgtcggactatgtaattgtaactatacctctggttcccattaaaagtgaccattttagttaaattttgttcctcttcccccttttcactttcctggaagatcgatgtcccgatcaggaaggtagagagttttcctgttcagattaccctgcccagcaggaactggaatacagtgttcggggagaaggccaaatgatatccttgagagcagagattaaacttttctgtcatggggaaagttggtgtataaatgagaaatgaagaacatgggatgtcatgagtgttggcctaaatttgcccagctatggggaatttttcctttaccacatttatttgcatactggtcttagtttatttgcagcagtttatccctttttaagaactctttgatcttttggcccttttaatggtgaggctcaaacaaactacatttaaatggggcagtattcagatttgaccatggtggagagcgcttagccactctgggtctttcacaggaaggagagtaactgagtgctgcaggagtttgtggagtggagtcaggatctaggaggtgagtgactcccttcctagctgccctggtgaacagcgcttgggtagatacctgctataaggagactggtctggctgggttactttcacatcctgcctgtactcagagggcttgaggtcattgacattatgagattttaggcttgatccctttttgattggagggtggaaggccctcctaagggaatgataagtgataagagggggaaggggttgcagccaatgagttaaaaccttagagcagtgctcctcagcctcttaccatgtggttgtaaacttgcacgtacctgccaaccagttatttagcatgctttttattttagttacacagagcgtaacattaacccaagagcagaaaggttttatttacagggttttcgaacttggtttgtaagacagctgccatcacaagcatagcttacaaatgtgctggggacccctaattgggaagtgctttcctctcaaatttttattttttatttttagagacagagtcttgctctgtcatccaggctggagtgcagtggcgtgatctcggctcactgtagcctctgcctcctgagtttaagcgattctcctgcctcaggagaatcccagcttctgagtagctgagactacaggcgtgggccaccatgcccacctagtttttgcatttttagtggaggtgtggtttcactgtgttggccaggctggtctcttaactcctgacctcaggtgatccacctgccttggcctcccaaagtgctggaattacaggcatgagccgctgcatctggccatcctctcaaattttcaagtgttccacaagtatgttctctactgaagagttgctgcatccttgaatcttgggtgatttgaggcacagaaactatgactttattttttgagatggagttttgctcttgttgcccaggctggagtgcaatggcacgtttttcggcttaccgcaactgccgcctcctgggttcaagcgatagctgggattacaggcatgcgccaccatgcccagctaatttatttgtatttttagtagagacggggtttctccatgttggtcaggctggtctcgaactcccgacctcaggtgatgtgcacacctcagcctcccaataaaccatgacttttaagaggaatagcaggtttacttcccctgccagcattggggtgctctctaagcaacagtaggcggagagtggtctggcgtattaaaaacaaaggatcgtcaagtgggccttcccaggcattgctttgacttagtacatgtagaggatgtggcagttctctccgtccctgccactgctggtttctttgttaaatgtttagttgaaatggcctgatacgatatttgagtagttcactgttggtgctttgcctagcaggattctaatcttgctttggttgtggtcccctgatgccctcctgttaggagtggaggaggtcgaagctccttgtaagatatgattactgggaccattagtgtcaagttcctgtgtccttcaaatggcatatgtgattggccttgaccttaaaaggaaatagggtcccaggtgactgtttagtgggtaggtccagtttggggggatcttccaggagaatggatagagacacctagcagcagagagaacattggtgcctctcaagccaacctcccacctcagcctcccaagtagctgggactacaggtgcttcctcgctaccacacctgggtaattttttttttaattacttttttttttttaagaaacagggtttcactgggttgcccaggctggtcttgaactggcctcaagtgacctgcctgcctcagcctcccaaagtgctgggatcacaggtgtgacccactgcgtttggccagaatactctattcttactgaatgattgaaatctgtcttgaagcattaggtgtcccatttttgtgagttggaattgggacaggctaagtaggaagtgaggagggtggggagagctgtgctgtaggtctgtttgtcccttccttgatgtagccttcagttagccctttcagcttttttccccatcttgtgccgggccttcctgggtttcagtacttggatgtagggctgcagttatgtcagtggtgggtagattgaccaggaattaaggtctagggtccagcccatgtgagacttgactcactgatctacctttaggcatgtcttccttccagtctcatcctttttaaattttttttttttttttgagacggtctcactctcacccaggctggattgcagtggtgtgatctcgaccaactgcaacctctgcctcccacccgcaagctatctgcccacctcagcctctggagtagctgggacgggactacaggcacctgccaccatgactggctaatttttttttgtattttttgtggagatggggtcttgccatgttgctcaggctggtctggtctcaaaactgctctgggctcaagtgattgtcccactttggcctcccagagtgctgggattaaggtgtgagatactgtgtccggctatgaaaattttatttttaattaacttgtatatatttatggggtacaatgtcctatttctgtacatgtacacattgtggaatcaaatcaggctaatatatccatcacttcatatcattagcatgaatgagaacatacaaagccactcttagaaaattttgaaatttatgttatttcagcccttttatgctggaggttgcaaatgttttgtgaataatcagaccaaaaataaaaacaaaaaatgattgacttcagtcattcagtaagaa

SEQ ID NO: 18 - DNA, 3707 bp, Artificial Sequence
  sequence delta 1-6 UTR
acttaagcttcgacgcaggaggcggggctgctcagtcctccaggcgtcggtactcagcggtgttggaacttcgttgcttgcttgcctgtgcgcgcgtgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccagcggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgcctggaagagggatgtaacattgccctgaggatggtgaagttggtatacatttataaagtacggaatggtgtcaatgaatgcaattctatgtatatggacttggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccactgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 19 - DNA, 1127 bp, Artificial Sequence
  intron 6 delta 1 et 2
gtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccag

SEQ ID NO: 20 - DNA, 894 bp, Artificial Sequence
  intron 7 delta 3  6
gtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgcctggaagagggatgtaacattgccctgaggatggtgaagttggtatacatttataaagtacggaatggtgtcaatgaatgcaattctatgtatatggacttggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccactgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaag

SEQ ID NO: 21 - DNA, 22 bp, Artificial Sequence
  primer hTLS FUS-For
gaattcgtggaccaggaaggtc

SEQ ID NO: 22 - DNA, 23 bp, Artificial Sequence
  primer hTLS FUS-Rev
cacgtgtgaactcaccggagtca

SEQ ID NO: 23 - DNA, 26 bp, Artificial Sequence
  primer FUS-For
gatttgaagtgggtagatagtgcagg

SEQ ID NO: 24 - DNA, 27 bp, Artificial Sequence
  primer FUS - Rev
cctttccacactttagtttagtcacag

SEQ ID NO: 25 - DNA, 19 bp, Artificial Sequence
  primer actin Fwd
ccaccagttcgccatggat

SEQ ID NO: 26 - DNA, 19 bp, Artificial Sequence
  primer actin rev
ggctttgcacatgccggag

SEQ ID NO: 27 - DNA, 22 bp, Artificial Sequence
  primer TBP fwd
ccaatgactcctatgaccccta

SEQ ID NO: 28 - DNA, 21 bp, Artificial Sequence
  primer TBP rev
cagccaagattcacggtagat

SEQ ID NO: 29 - DNA, 18 bp, Artificial Sequence
  primer Pol2 fwd
gctgggagacatagacca

SEQ ID NO: 30 - DNA, 20 bp, Artificial Sequence
  primer Pol2 rev
ttactcccctgcatggtctc

SEQ ID NO: 31 - DNA, 20 bp, Artificial Sequence
  primer hFUS fwd
gccagaacacaggctatgga

SEQ ID NO: 32 - DNA, 20 bp, Artificial Sequence
  primer hFUS rev
cgattgggagctctggctac

SEQ ID NO: 33 - DNA, 23 bp, Artificial Sequence
  primer T-Fus fwd
ttatggacagacccaaaaacaca

SEQ ID NO: 34 - DNA, 20 bp, Artificial Sequence
  primer T-Fus rev
tgctgcccataagaagattg

SEQ ID NO: 35 - DNA, 24 bp, Artificial Sequence
  primer Fus delta NLS fwd
gcaagatggactcctagtgttaat

SEQ ID NO: 36 - DNA, 22 bp, Artificial Sequence
  primer FUS delat NLS rev
acctctacaaatgtggtatggc

SEQ ID NO: 37 - DNA, 17 bp, Artificial Sequence
  primer Fus exon 6-8 fwd
cggcatggggtcctcgg

SEQ ID NO: 38 - DNA, 20 bp, Artificial Sequence
  primer Fus exon 6-8 rev
cctaggccttgcacgaagat

SEQ ID NO: 39 - protein, 9 aa, Artificial Sequence
  HA tag
YPYDVPDYA

SEQ ID NO: 40 - protein, 10 aa, Artificial Sequence
  myc tag
EQKLISEEDL

SEQ ID NO: 41 - protein, 8 aa, Artificial Sequence
  Flag tag
DYKDDDDK

SEQ ID NO: 42 - DNA, 2545 bp, Artificial Sequence
  HA-E1-E2-E3-E4-E5-E6-1-E7-E8-E9-E10-E11-E12-E13-E14-E15
acttaagcttcgacgcaggaggcggggctgctcagtcctccaggcgtcggtactcagcggtgttggaacttcgttgcttgcttgcctgtgcgcgcgtgcgcggacatgtacccatacgatgttccagattacgctgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattctttcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggaggatgtattttagtagccacttgtatctttttccaaaaacacctacccatgtttggggaatgttaaacaaaatcaaaaaacaaccttttgtagccgttggaagcttcatgtcctttcttctaacttgtcttctccacggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagtttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 43 - DNA, 2347 bp, Artificial Sequence
  HA-E1-E2-E3-E4-E5-E6-2-E7-E8-E9-E10-E11-E12-E13-E14-E15
acttaagcttcgacgcaggaggcggggctgctcagtcctccaggcgtcggtactcagcggtgttggaacttcgttgcttgcttgcctgtgcgcgcgtgcgcggacatgtacccatacgatgttccagattacgctgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgggatgtattttagtagccacttgtatctttttccaaaaacacctacccatgtttggggaatgttaaacaaaatcaaaaaacaaccttttgtagccgttggaagcttcatgtcctttcttctaacttgtcttctccacggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagtttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 44 - DNA, 2347 bp, Artificial Sequence
  HA-E1-E2-E3-E4-E5-E6-E7-3-E8-E9-E10-E11-E12-E13-E14-E15
acttaagcttcgacgcaggaggcggggctgctcagtcctccaggcgtcggtactcagcggtgttggaacttcgttgcttgcttgcctgtgcgcgcgtgcgcggacatgtacccatacgatgttccagattacgctgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttggatgtattttagtagccacttgtatctttttccaaaaacacctacccatgtttggggaatgttaaacaaaatcaaaaaacaaccttttgtagccgttggaagcttcatgtcctttcttctaacttgtcttctccacggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 45 - DNA, 2347 bp, Artificial Sequence
  HA-E1-E2-E3-E4-E5-E6-E7-4-E8-E9-E10-E11-E12-E13-E14-E15
acttaagcttcgacgcaggaggcggggctgctcagtcctccaggcgtcggtactcagcggtgttggaacttcgttgcttgcttgcctgtgcgcgcgtgcgcggacatgtacccatacgatgttccagattacgctgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttggatgtattttagtagccacttgtatctttttccaaaaacacctacccatgtttggggaatgttaaacaaaatcaaaaaacaaccttttgtagccgttggaagcttcatgtcctttcttctaacttgtcttctccacggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagtaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 46 - DNA, 2347 bp, Artificial Sequence
  HA-E1-E2-E3-E4-E5-E6-E7-5-E8-E9-E10-E11-E12-E13-E14-E15
acttaagcttcgacgcaggaggcggggctgctcagtcctccaggcgtcggtactcagcggtgttggaacttcgttgcttgcttgcctgtgcgcgcgtgcgcggacatgtacccatacgatgttccagattacgctgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttggatgtattttagtagccacttgtatctttttccaaaaacacctacccatgtttggggaatgttaaacaaaatcaaaaaacaaccttttgtagccgttggaagcttcatgtcctttcttctaacttgtcttctccacggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttcttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 47 - DNA, 2347 bp, Artificial Sequence
  HA-E1-E2-E3-E4-E5-E6-E7-6-E8-E9-E10-E11-E12-E13-E14-E15
acttaagcttcgacgcaggaggcggggctgctcagtcctccaggcgtcggtactcagcggtgttggaacttcgttgcttgcttgcctgtgcgcgcgtgcgcggacatgtacccatacgatgttccagattacgctgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttggatgtattttagtagccacttgtatctttttccaaaaacacctacccatgtttggggaatgttaaacaaaatcaaaaaacaaccttttgtagccgttggaagcttcatgtcctttcttctaacttgtcttctccacggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagtctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctccttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 48 - DNA, 3535 bp, Artificial Sequence
  HA-E1-E2-E3-E4-E5-E6-1-2-E7-3-4-5-6-E8-E9-E10-E11-E12-E13-E14-E15
acttaagcttcgacgcaggaggcggggctgctcagtcctccaggcgtcggtactcagcggtgttggaacttcgttgcttgcttgcctgtgcgcgcgtgcgcggacatgtacccatacgatgttccagattacgctgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattctttcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggattatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgggatgtattttagtagccacttgtatctttttccaaaaacacctacccatgtttggggaatgttaaacaaaatcaaaaaacaaccttttgtagccgttggaagcttcatgtcctttcttctaacttgtcttctccacggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttcttctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctccttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 49 - DNA, 2743 bp, Artificial Sequence
  HA-E1-E2-E3-E4-E5-E6-1-2-E7-E8-E9-E10-E11-E12-E13-E14-E15
acttaagcttcgacgcaggaggcggggctgctcagtcctccaggcgtcggtactcagcggtgttggaacttcgttgcttgcttgcctgtgcgcgcgtgcgcggacatgtacccatacgatgttccagattacgctgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattctttcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggattatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgggatgtattttagtagccacttgtatctttttccaaaaacacctacccatgtttggggaatgttaaacaaaatcaaaaaacaaccttttgtagccgttggaagcttcatgtcctttcttctaacttgtcttctccacggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagtttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 50 - DNA, 2941 bp, Artificial Sequence
  HA-E1-E2-E3-E4-E5-E6-E7-3-4-5-6-E8-E9-E10-E11-E12-E13-E14-E15
acttaagcttcgacgcaggaggcggggctgctcagtcctccaggcgtcggtactcagcggtgttggaacttcgttgcttgcttgcctgtgcgcgcgtgcgcggacatgtacccatacgatgttccagattacgctgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttggatgtattttagtagccacttgtatctttttccaaaaacacctacccatgtttggggaatgttaaacaaaatcaaaaaacaaccttttgtagccgttggaagcttcatgtcctttcttctaacttgtcttctccacggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttcttctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctccttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 51 - DNA, 3535 bp, Artificial Sequence
  HA-E1-E2-E3-E4-E5-E6-1-2-E7-3-4-5-6-E8-E9-E10-E11-E12-E13-E14-E15
acttaagcttcgacgcaggaggcggggctgctcagtcctccaggcgtcggtactcagcggtgttggaacttcgttgcttgcttgcctgtgcgcgcgtgcgcggacatgtacccatacgatgttccagattacgctgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttcttctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctggatgtattttagtagccacttgtatctttttccaaaaacacctacccatgtttggggaatgttaaacaaaatcaaaaaacaaccttttgtagccgttggaagcttcatgtcctttcttctaacttgtcttctccacggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagtcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggattatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgtttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 52 - DNA, 2941 bp, Artificial Sequence
  HA-E1-E2-E3-E4-E5-E6-3-4-E7-5-6-E8-E9-E10-E11-E12-E13-E14-E15
acttaagcttcgacgcaggaggcggggctgctcagtcctccaggcgtcggtactcagcggtgttggaacttcgttgcttgcttgcctgtgcgcgcgtgcgcggacatgtacccatacgatgttccagattacgctgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagcggatgtattttagtagccacttgtatctttttccaaaaacacctacccatgtttggggaatgttaaacaaaatcaaaaaacaaccttttgtagccgttggaagcttcatgtcctttcttctaacttgtcttctccacggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttcttctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctccttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 53 - DNA, 3535 bp, Artificial Sequence
  HA-E1-E2-E3-E4-E5-E6-3-4-1-E7-5-6-2-E8-E9-E10-E11-E12-E13-E14-E15
acttaagcttcgacgcaggaggcggggctgctcagtcctccaggcgtcggtactcagcggtgttggaacttcgttgcttgcttgcctgtgcgcgcgtgcgcggacatgtacccatacgatgttccagattacgctgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggaggatgtattttagtagccacttgtatctttttccaaaaacacctacccatgtttggggaatgttaaacaaaatcaaaaaacaaccttttgtagccgttggaagcttcatgtcctttcttctaacttgtcttctccacggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttcttctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgtttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 54 - DNA, 396 bp, Artificial Sequence
  intron 6 motif 1
tcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggagga

SEQ ID NO: 55 - DNA, 198 bp, Artificial Sequence
  intron 6 motif 2
ttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctg

SEQ ID NO: 56 - DNA, 198 bp, Artificial Sequence
  intron 7 motif 3
ccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattc

SEQ ID NO: 57 - DNA, 198 bp, Artificial Sequence
  intron 7 motif 4
taactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagc

SEQ ID NO: 58 - DNA, 198 bp, Artificial Sequence
  intron 7 motif 5
tacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttct

SEQ ID NO: 59 - DNA, 198 bp, Artificial Sequence
  intron 7 motif 6
tctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcct

SEQ ID NO: 60 - DNA, 20 bp, Artificial Sequence
  Primer
gggcatgggtcagaaggatt

SEQ ID NO: 61 - DNA, 20 bp, Artificial Sequence
  Primer
tcgatggggtacttcagggt

SEQ ID NO: 62 - DNA, 20 bp, Artificial Sequence
  Primer
ttcaccaccatggagaaggc

SEQ ID NO: 63 - DNA, 20 bp, Artificial Sequence
  Primer
agagggggcagagatgatga

SEQ ID NO: 64 - DNA, 20 bp, Artificial Sequence
  Primer
ttgctttccttggtcaggca

SEQ ID NO: 65 - DNA, 20 bp, Artificial Sequence
  Primer
atccaacacttcgtggggtc

SEQ ID NO: 66 - DNA, 20 bp, Artificial Sequence
  Primer
ccgttggaagcttcatgtcc

SEQ ID NO: 67 - DNA, 21 bp, Artificial Sequence
  Primer
tattgaagccaccacggtcac

SEQ ID NO: 68 - DNA, 24 bp, Artificial Sequence
  Primer
ctgtgagcacttacttgatatttt

SEQ ID NO: 69 - DNA, 17 bp, Artificial Sequence
  Primer
gtgatccttggtcccga

(19) United States
(12) Patent Application Publication
(10) Pub. No.: US 2024/0024513 A1
(43) Pub. Date: Jan. 25, 2024

(54) NUCLEIC ACIDS ENCODING HUMAN FUS PROTEIN AND USE IN THE TREATMENT OF AMYOTROPHIC LATERAL SCLEROSIS (ALS)

(71) Applicants: INSERM - Institut National de la Santé et de la Recherche Médicale, Paris (FR); Université de Strasbourg, Strasbourg (FR)

(72) Inventors: Luc Dupuis, Strasbourg (FR); Immaculada Sanjuan Ruiz, Strasbourg (FR); Gina Picchiarelli, Strasbourg (FR)

(21) Appl. No.: 17/915,569

(22) PCT Filed: Apr. 9, 2021
(86) PCT No.: PCT/EP2021/059322

§ 371 Date: Sep. 29, 2022

(30) Foreign Application Priority Data
Apr. 9, 2020 (EP) 20315141.0

(51) Int. Cl. A61K 48/00; C07K 14/47; C12N 15/86; A61P 25/02; A61P 25/28

(52) U.S. Cl. CPC A61K 48/0066; C07K 14/4702; C12N 15/86; A61P 25/02; A61P 25/28

(57) ABSTRACT

The invention relates to a nucleic acid encoding human FUS protein comprising a sequence having at least 69% sequence identity with the sequence SEQ ID NO 1 of intron 6 and/or a sequence having at least 60% sequence identity with the sequence SEQ ID NO 2 of intron 7, said sequences being located on either side of exon 7, to a recombinant vector comprising said nucleic acid encoding human FUS protein, and their use as medicament in the treatment and/or prevention of amyotrophic lateral sclerosis (ALS). The invention further relates to a pharmaceutical composition comprising said nucleic acid and/or vector for the prevention and/or treatment of amyotrophic lateral sclerosis (ALS). The present invention finds application in the therapeutic, veterinary and diagnostic medical technical fields.

### FIELD

The invention relates to a nucleic acid encoding human FUS protein comprising a sequence having at least 69% sequence identity with the sequence SEQ ID NO 1 of intron 6 and/or a sequence having at least 60% sequence identity with the sequence SEQ ID NO 2 of intron 7, said sequences being located on either side of exon 7, to a recombinant vector comprising said nucleic acid encoding human FUS protein, and their use as medicament in the treatment and/or prevention of amyotrophic lateral sclerosis (ALS). The invention also relates to a host cell comprising said nucleic acid and/or vector. The invention further relates to a pharmaceutical composition comprising said nucleic acid and/or vector for the prevention and/or treatment of amyotrophic lateral sclerosis (ALS).

The present invention finds application in the therapeutic, veterinary and diagnostic medical technical fields.

In the description below, references in square brackets ([ ]) refer to the list of references at the end of the text.

### BACKGROUND

Amyotrophic lateral sclerosis (ALS), also cited as motor neuron disease (MND) or Lou Gehrig's disease, involves degeneration of motor neurons in the brainstem, spinal cord and motor cortex, with onset around 60 years of age. The consequence of motor neuron loss includes muscle weakness and muscle atrophy. Depending on site of onset, the primary symptoms affect upper limbs, lower limbs or muscles of the face and neck. In other words, people who suffer from Amyotrophic lateral sclerosis (ALS) are losing progressively their capacity eliciting efficient movements, and this culminates to loss of respiratory capacity causing death.

The cause and the biological mechanisms involved in amyotrophic lateral sclerosis (ALS) are not known. However, it appears that this disease involves both genetic and environmental contributions. In particular, a subset of ALS cases is dominantly inherited, demonstrating a genetic contribution, in these so-called familial ALS cases (fALS). fALS patients develop usually motor symptoms earlier, and life expectancy is shorter than non familial (so-called sporadic) patients. Most of the affected people develop their motor symptoms at about 50-60 years of age, yet there are cases with onset below 40 years of age. In Europe, the disease affects about two to three new people per 100,000 per year.

Once affected, the mean survival time is about 2 to 6 years. To date, there is no treatment and/or any mean for treating the etiological causes of amyotrophic lateral sclerosis (ALS). Only two treatments are currently approved by the regulatory agencies. Riluzole has a modest effect in prolonging lifespan, yet does not ameliorate function, while edaravone has shown moderate protective activity in a small subset of patients. ALS patients also undergo a number of treatments to manage the progression of symptoms, that can be considered as comfort care. A reason for the failure to develop new efficient drugs might rely on the fact that ALS is rather a compendium of multiple diseases with completely different causes and mechanisms converging to similar clinical picture in end stage. It is thus highly needed to develop new therapeutic strategies, for example which target etiological mechanisms.

There is therefore a real need for a compound and/or method overcoming the shortcomings, disadvantages and obstacles of prior art, particularly for a compound and or method for treating and/or preventing the amyotrophic lateral sclerosis (ALS).

### DESCRIPTION

The present invention allows to overcome the drawback and inconvenient of the prior art by providing a nucleic acid encoding human FUS protein comprising: a sequence having at least 69% sequence identity with the sequence SEQ ID NO 1 of intron 6 and/or a sequence having at least 60% sequence identity with the sequence SEQ ID NO 2 of intron 7 and/or at least one preserved RNA motif within sequence SEQ ID NO 1 or SEQ ID NO 2 able to bind human FUS protein.

The inventors have surprisingly and unexpectedly demonstrated that a nucleic acid according to the invention are useful to treat people suffering from ALS. In particular, the inventors have surprisingly demonstrated that the nucleic acid of the present invention allows to treat and cure amyotrophic lateral sclerosis (ALS), in particular amyotrophic lateral sclerosis (ALS), from patients affected by severe juvenile forms of ALS related to mutations in the FUS gene.

In particular, the inventors have surprisingly demonstrated that the invention is able to simultaneously decrease the mutant protein and replete in normal, physiologically active wild type protein while avoiding excess toxic production of the wild type protein.

In particular, it is known that mutations in FUS gene lead to cytoplasmic accumulation of the physiologically nuclear mutated FUS protein. Cytoplasmic accumulation of FUS is a widespread phenomenon in most ALS cases (Tyzack 2019) and in a large subset of cases affected with fronto-temporal dementia (Urwin 2010), although not associated with germline mutations. In cases with germline mutations, it has been demonstrated that the toxicity of the mutation comes from both accumulation of cytoplasmic FUS, and simultaneous loss of nuclear FUS. Indeed, the toxicity of cytoplasmic FUS has been repeatedly demonstrated (eg Scekic-Zahirovic EMBO J 2016), and loss of nuclear FUS leads to a number of transcriptional abnormalities which are overall toxic for neurons and synapses.

The inventors have demonstrated that an efficient treatment for ALS mediated by FUS mutations should not only correct loss of nuclear function, but also prevent increased cytoplasmic accumulation. A simple overexpression of FUS protein, through cDNA driven overexpression systems, could in principle rescue loss of function, but would not be expected to avoid accumulation of toxic cytoplasmic FUS from mutant alleles, and, most importantly has been shown to be highly toxic to neurons (Mitchell J C, 2013 “Overexpression of human wild-type FUS causes progressive motor neuron degeneration in an age- and dose-dependent fashion” Acta Neuropathologica volume 125, pages 273-288(2013 DOI: 10.1007/s00401-012-1043-z [12]).

The inventors have surprisingly demonstrated that the nucleic acid encoding human FUS gene, preferably the full length nucleic acid encoding human FUS gene, is able to synergistically prevent the cytoplasmic accumulation of mutated FUS, restore FUS in the nucleus, without any toxicity. In particular, the present invention, does not induce any toxicity contrary to the classically used overexpression systems.

The inventors have surprisingly demonstrated that providing the nucleic acid is sufficient to fully rescue the perinatal lethality of mice carrying homologous Fus mutation (FIG. 1), and rescue the motor defect observed in heterozygous Fus mutant mice (FIG. 2). Surprisingly and advantageously, the rescue was associated with a complete reversal of cytoplasmic FUS accumulation, as judged from biochemical fractionation of nuclear and cytoplasmic fractions, immunohistochemistry and immunofluorescence (FIG. 3). It is particularly striking and surprising that no toxicity was observed in treated transgenic mice until two years of age as wild type FUS protein overexpression driven by cDNA is by itself highly toxic (Mitchell J C, 2013 “Overexpression of human wild-type FUS causes progressive motor neuron degeneration in an age- and dose-dependent fashion” Acta Neuropathologica volume 125, pages 273-288(2013 DOI: 10.1007/s00401-012-1043-z [12]). The inventors demonstrated that no toxicity is due to the fact the provided nucleic acid include genomic constructs, allowing for autoregulation of the FUS gene and avoiding the deleterious effects of uncontrolled FUS levels.

In the present invention, the nucleic acid encoding human FUS protein according to the invention may comprise a nucleic acid sequence having at least 69% 70%, 75%, 80%, 85%, 90%, 91%, 92%, 93%, 94%, 95%, 96%, 97%, 98%, 99%, 99.5% or 100% sequence identity with the sequence SEQ ID NO 1 of intron 6. It may be, for example, a nucleic acid of sequence SEQ ID NO 3, SEQ ID NO 4 or SEQ ID NO. 19.

In the present invention, when the nucleic acid encoding human FUS protein according to the invention comprises a nucleic acid sequence having at least 69% 70%, 75%, 80%, 85%, 90%, 91%, 92%, 93%, 94%, 95%, 96%, 97%, 98%, 99%, 99.5% or 100% sequence identity with the sequence SEQ ID NO 1 of intron 6, said sequence having an identity with the sequence SEQ ID NO 1 of intron 6 may be located before exon 7.

In the present invention, the nucleic acid encoding human FUS protein according to the invention may comprise a nucleic acid sequence having at least 60%, 65%, 70%, 75%, 80%, 85%, 90%, 91%, 92%, 93%, 94%, 95%, 96%, 97%, 98%, 99%, 99.5% or 100% sequence identity with the sequence SEQ ID NO 2. It may be, for example, a nucleic acid of sequence SEQ ID NO 5, SEQ ID NO 6, SEQ ID NO 7, SEQ ID NO 8 or SEQ ID NO 20.

In the present invention, when the nucleic acid encoding human FUS protein according to the invention comprises a nucleic acid sequence having at least 60%, 65%, 70%, 75%, 80%, 85%, 90%, 91%, 92%, 93%, 94%, 95%, 96%, 97%, 98%, 99%, 99.5% or 100% sequence identity with the sequence SEQ ID NO 2, said sequence having an identity with the sequence SEQ ID NO 2 may be located after exon 7.

In the present invention, the nucleic acid encoding human FUS protein according to the invention may comprise at least one preserved RNA motif within sequence SEQ ID NO 1 or SEQ ID NO 2 able to bind human FUS protein. Advantageously, the at least one preserved RNA motif may be located before or after exon 7. If more than one preserved RNA motif is present, at least one preserved RNA motif may be located before exon 7, and at least one preserved RNA motif may be located after exon 7. A motif may be a nucleic acid of having a sequence of about 180 nucleic acids to about 450 nucleic acids. It may be, for example, a nucleic acid having a sequence of about 198 to 396 nucleic acid. For example, when the motif is within sequence SEQ ID NO 1 of intron 6, it may be selected from the group consisting of the sequences SEQ ID NO 54 and SEQ ID NO 55, or a sequence having at least 90% identity with said sequences as long as the sequence retain the ability to bind human FUS protein. When the motif is within sequence SEQ ID NO 2 of intron 7, it may be selected from the group consisting of the sequences SEQ ID NO 56, SEQ ID NO 57, SEQ ID NO 58 and SEQ ID NO 59, or a sequence having at least 90% identity with said sequences as long as the sequence retain the ability to bind human FUS protein. The number of motifs in the nucleic acid of the invention may be 1, or 2, or 3, or 4, or 5, or 6, or 7, or 8, or 9, or 10, or more. Advantageously, the number of motifs in the nucleic acid of the invention is comprised between 1 and 6.

Advantageously, the nucleic acid encoding human FUS protein according to the invention may comprise in addition to the RNA motifs to which FUS binds, some sequences on both sides of the exons, for example 99 nucleotides at the beginning of intron 6, and/or 136 nucleotides at the end of intron 6, and/or 76 nucleotides at the beginning of intron 7 and/or 125 nucleotides at the end of intron 7. Without wanting to be bound by a particular mechanism of action, these sequences may be regulatory sequences of FUS that may be of importance for its autoregulation.

In the present invention a nucleic acid encoding human FUS protein according to the invention may comprise at least one sequence selected among a sequence having at least 69% sequence identity with the sequence SEQ ID NO 1 of intron 6, a sequence having at least 60% sequence identity with the sequence SEQ ID NO 2 of intron 7, and at least one preserved RNA motif within sequence SEQ ID NO 1 or SEQ ID NO 2 able to bind human FUS protein, said at least one sequence being located on either side of exon 7.

In the present invention a nucleic acid encoding human FUS protein according to the invention may comprise a sequence having at least 69%, 70%, 75%, 80%, 85%, 90%, 91%, 92%, 93%, 94%, 95%, 96%, 97%, 98%, 99%, 99.5% or 100% sequence identity with the sequence SEQ ID NO 1 of intron 6 and a sequence having least 60%, 65%, 70%, 75%, 80%, 85%, 90%, 91%, 92%, 93%, 94%, 95%, 96%, 97%, 98%, 99%, 99.5% or 100% sequence identity with the sequence SEQ ID NO 2 of intron 7, and at least one preserved RNA motif within sequence SEQ ID NO 1 or SEQ ID NO 2 able to bind human FUS protein, said sequences being located on either side of exon 7. It may be, for example, a nucleic acid encoding human FUS protein of sequence SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52 and SEQ ID NO 53.

In the present invention, the localization of the nucleic acid sequence, for example sequence SEQ ID NO 1 and/or 2, in the nucleic acid encoding human FUS protein may refers to the position of the sequence from the 5′ to 3′ direction.

In the present invention, gene encoding human FUS means the non mutated and/or wild-type human gene of FUS. I sequence SEQ ID NO 10 located on chromosome 16 (Gene ID: 2521). The term “wild-type”, as used herein, refers to a gene or gene product that has the characteristics of that gene or gene product when isolated from a naturally-occurring source. A wild-type gene or gene product (e.g., a polypeptide) is that which is most frequently observed in a population and is thus arbitrarily designed the “normal” or “wild-type” form of the gene.

In the present invention, mutated gene encoding human FUS means any form of the gene encoding human FUS protein carrying a primary sequence with any variations as compared to the wild type form. It may be any mutation that could lead to a mutated human FUS protein, for example a human protein FUS having mutations in the C terminal region of the protein, for example associated with a disease.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

In the present, the term “isolated” and its grammatical equivalents as used herein refer to the removal of a nucleic acid from its natural environment.

In the present, the term “purified” and its grammatical equivalents as used herein refer to a molecule or composition, whether removed from nature (including genomic DNA and mRNA) or synthesized (including cDNA) and/or amplified under laboratory conditions, that has been increased in purity, wherein “purity” is a relative term, not “absolute purity.” It is to be understood, however, that nucleic acids and proteins can be formulated with diluents or adjuvants and still for practical purposes be isolated. For example, nucleic acids may be mixed with an acceptable carrier or diluent when used for introduction into cells.

In the present, the term “substantially purified” and its grammatical equivalents as used herein refer to a nucleic acid sequence, polypeptide, protein or other compound which is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

In the present, “polynucleotide(s)”, “oligonucleotide(s)”, “nucleic acid(s)”, “nucleotide(s)”, “polynucleic acid(s)”, or any grammatical equivalent as used herein refers to a polymeric form of nucleotides or nucleic acids of any length, either ribonucleotides or deoxyribonucleotides. This term refers only to the primary structure of the molecule. Thus, this term includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs. The nucleic acid sequences and vectors disclosed or contemplated herein can be introduced into a cell by, for example, transfection, transformation, or transduction.

In the present “transfection,” “transformation,” or “transduction” refer to the introduction of one or more exogenous polynucleotides into a host cell by using physical or chemical methods. It may be any transfection and/or transformation and/or transduction adapted method known to one skilled in the art. It may be for example, calcium phosphate DNA co-precipitation (see, e.g., Murray E. J. (ed.), Methods in Molecular Biology, Vol. 7, Gene Transfer and Expression Protocols, Humana Press (1991) [1]); DEAE-dextran; electroporation; cationic liposome-mediated transfection; tungsten particle-facilitated microparticle bombardment (Johnston, Nature, 346: 776-777 (1990) [2]); and strontium phosphate DNA co-precipitation (Brash et al., Mol. Cell Biol., 7: 2031-2034 (1987)). Phage, viral, or non-viral vectors can be introduced into host cells, after growth of infectious particles in suitable packaging cells, many of which are commercially available. In some embodiments, lipofection, nucleofection, or temporary membrane disruption (e.g., electroporation or deformation) can be used to introduce one or more exogenous polynucleotides into the host cell.

In the present, “preserved RNA motif” as used herein refers to a sequence that presents identical sequence with the Human FUS gene and able to bind on the Human FUS protein.

In the present “polypeptide”, “peptide” and their grammatical equivalents as used herein refer to a polymer of amino acid residues.

In the present a “functional protein” is a protein which is biologically active and which, optionally, comprises glycosylation or other modifications typical for the protein in a given cellular environment.

Polypeptides and proteins disclosed herein (including functional portions and functional variants thereof) can comprise synthetic amino acids in place of one or more naturally-occurring amino acids. It may be any synthetic amino acids known to one skilled in the art, it may be for example aminocyclohexane carboxylic acid, norleucine, a-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′, N′-dibenzyl-lysine, 6-hydroxylysine, ornithine, a-aminocyclopentane carboxylic acid, a-aminocyclohexane carboxylic acid, a-aminocycloheptane carboxylic acid, a-(2-amino-2-norbornane)-carboxylic acid, α,γ-diaminobutyric acid, α,β-diaminopropionic acid, homophenylalanine, and a-tert-butylglycine.

In the present polypeptides and proteins may comprise any post-translational modifications known to one skilled in the art. It may comprise, for example one or more amino acids with post-translational modification. The post-translational modification of one or more amino acids may be for example phosphorylation, acylation including acetylation and formylation, glycosylation (including N-linked and O-linked), amidation, hydroxylation, alkylation including methylation and ethylation, ubiquitylation, addition of pyrrolidone carboxylic acid, formation of disulfide bridges, sulfation, myristoylation, palmitoylation, isoprenylation, farnesylation, geranylation, glypiation, lipoylation and iodination.

In the present, nucleic acids and/or nucleic acid sequences may be considered as “homologous” when they are derived, naturally or artificially, from a nucleic acid sequence.

In the present, proteins and/or protein sequences are “homologous” when their encoding DNAs are derived, naturally or artificially, from a nucleic acid sequence. For example, protein as described herein may be modified by any available adapted mutagenesis method known to one skilled in the art. For example when expressed, the mutagenized nucleic acid encodes a polypeptide that is homologous to the protein encoded by the original nucleic acid. Homology is generally inferred from sequence identity between two or more nucleic acids or proteins (or sequences thereof). The precise percentage of identity between sequences that is useful in establishing homology varies with the nucleic acid and protein at issue, it may be for example a percentage of sequence identity of at least 25%, for example at least 30%, 40%, 50%, 60%, 70%, 80%, 90%, 95% or 99%.

The percentage of sequence identity may be determined by any method known to one skilled in the art. It may be determined for example by use of BLASTP and BLASTN, for example using default parameters.

In the present homologous molecules may be also termed homologs.

In the present, the terms “identical” and its grammatical equivalents as used herein and/or “sequence identity” in the context of two nucleic acid sequences or of two amino acid sequences of polypeptides refers to the residues in a sequence which are the same when aligned for maximum correspondence over a sequence length of at least 20 contiguous nucleic acid or amino acids. It may be for example two sequences which are the same when aligned for maximum correspondence over a sequence length of at least about 50 at least about 200 contiguous nucleic acid or amino acids. The identity of the sequence may be determined by comparing a sequence to a reference sequence of the same number of contiguous positions after the two sequences are aligned optimally.

The determination of the identity and/or the comparison of a sequence to a reference sequence may be carried out with any methods and/or process adapted known to one skilled in the art. It may be for example a method of alignment of sequences for comparison, for example a method of alignment of sequences using the local homology algorithm of Smith and Waterman, Adv. Appl. Math., 2:482 (1981) [4]; using the alignment algorithm of Needleman and Wunsch, J. Mol. Biol., 48:443 (1970) [5]; using a search for similarity method of Pearson and Lipman, Proc. Nat. Acad. Sci U.S.A., 85:2444 (1988) [6]; using a computerized implementations of these algorithms, for example CLUSTAL in the PC/Gene program by Intelligences, Mountain View Calif, GAP, BESTFIT, BLAST, FASTA, and TFASTA in the Wisconsin Genetics Software Package, Genetics Computer Group (GCG), 575 Science Dr., Madison, Wis., U.S.A.) Alignment is also often performed by inspection and manual alignment.

In the present, the nucleic acid may be produced and/or obtained by any method adapted known from one skilled in the art. For example, the nucleic acid may be synthesized using the BigDye™ Terminator v3.1 Cycle Sequencing Kit (thermofisher, 4337454).

Another object of the present invention is a recombinant vector comprising a nucleic acid encoding human FUS protein comprising a sequence having at least 69% sequence identity with the sequence SEQ ID NO 1 of intron 6 and/or a sequence having at least 60% sequence identity with the sequence SEQ ID NO 2 of intron 7 and/or at least one preserved RNA motif within sequence SEQ ID NO 1 or SEQ ID NO 2 able to bind human FUS protein, as explained above.

According to the invention, the vector may be any vector known from skilled in the art adapted to the expression of a nucleic acid. It may be for example any vector selected from the vectors listed in the catalogue http://www.promega.com/vectors/mammalian_express_vectors.htm [10] or http://www.qiagen.com/pendantview/qiagenes.aspx?gaw=PROTQIAgenes 0807&gkw=mammalian+expression [11], or else http://www.scbt.com/chap _exp_vectors.php?type=pCruzTM%20Expression%20Vectors [12]. It may, for example, be the expression vector described in document WO 83/004261 [13]. The vector may be, for example Adeno-associated Virus (AAV) vectors, a plasmid, a Yeast Artificial Chromosomes (YAC) or a Bacterial Artificial Chromosome (BAC).

The vector may be any adapted adeno-associated virus (AAV) vector known to one skilled in the art and/or commercially available adapted. It may be, for example any adapted AAV disclosed in Naso et al 2017, “Adeno-Associated Virus (AAV) as a Vector for Gene Therapy”, BioDrugs. 2017 August; 31(4):317-334. doi: 10.1007/s40259-017-0234-5 and/or in Zincarelli 2008 “Analysis of AAV Serotypes 1-9 Mediated Gene Expression and Tropism in Mice After Systemic Injection” Mol Ther. 2008 June; 16(6):1073-80. doi: 10.1038/mt.2008.76. Epub 2008 Apr. 15. It may be, for example an adeno-associated virus (AAV) vector selected from the group comprising AAV1, AAV2, AAV3, AAV4, AAV5, AAV6, AAV7, AAV8, AAV9 or AAV10 (AAVrh.10). preferably AAV9.

The vector may be any adapted plasmid known to one skilled in the art and/or commercially available. It may be for example a plasmid selected from the group comprising pMX, pUC19, pHP45-CmR, pcDNA3.1(+), pcDNA3.3-TOPO, pcDNA3.4-TOPO, pFastBac1, pET100/D-TOPO, pET151/D-TOPO, pRSET A, pYes2.1V5-His TOPO, pDONR221, pGEX-3X, preferably pcDNA3.1(+).

The vector may be any adapted Yeast Artificial Chromosome known to one skilled in the art. It may be for example a Yeast Artificial Chromosomes selected from the group comprising pYAC-RC, pYAC3

The vector may be any adapted Bacterial Artificial Chromosome known to one skilled in the art. It may be for example a Bacterial Artificial Chromosome selected from the group comprising pUvBBAC, pCC1BAC, pBAC 108L

The vector may comprise a polynucleotide sequence, for example an expression cassette, comprising the following in 5′ to 3′order:

a promoter sequence;

a nucleic acid sequence encoding a human FUS of the invention; and

a polyadenylation (polyA) sequence.

The promoter may be any promoter know to one skilled in the art. It may be for example promoters of housekeeping gene, promoters of viral gene, tissue specific promoter, promoters targeting neurons, promoters targeting muscle.

It may be for example promoters of housekeeping genes selected from the group comprising

It may be for example promoters of viral genes selected from the group comprising CMV promoter.

It may be for example a tissue specific promoter, for example a neural tissue and/or neural cell specific promoter. It may be for example a promoter selected from the group comprising NSE, Camk2a, Thy1, Fezf2, Crym promoter. It may be for example a tissue specific promoter, for example a muscle tissue and/or muscle cell specific promoter. It may be for example a promoter selected from the group comprising myoD, myf5, MyoG, Desmin promoters.

The vector may further comprise a nucleic acid sequence encoding for tag protein. It may be for example any nucleic acid sequence encoding for tag protein know to one skilled in the art. For example, it may be any tag disclosed in Parkinson J1, Blaxter M. “Expressed sequence tags: an overview.” Methods Mol Biol. 2009; 533:1-12. doi: 10.1007/978-1-60327-136-3_1.

The vector may further comprise additional sequence that may selectively label transgenic protein, for example aka tags, for example sequences allowing the production of the HA (sequence: YPYDVPDYA SEQ ID NO 39), myc (EQKLISEEDL SEQ ID NO 40) or FLAG (DYKDDDDK SEQ ID NO 41) tags

The vector may be selected according to the selected host cell. One skilled in the art, taking into consideration its technical knowledge, will adapt the vector in light of the host cell.

The host cell may be any host suitable for the expression of the nucleic acids or the vectors of the invention. It may, for example, be mammalian cells, E. coli, Pischia pastoris, Saccharomyces cerevisiae, or insect cells, for example an insect cell-baculovirus system, for example SF9 insect cells used in a baculovirus expression system.

Another object of the present invention is a nucleic acid and/or a recombinant vector and/or host cell for use as a medicament in the treatment and/or prevention of amyotrophic lateral sclerosis (ALS).

The nucleic acid for use as a medicament in the treatment and/or prevention of amyotrophic lateral sclerosis (ALS) may be any nucleic acid of the invention as defined above.

The vector for use as a medicament in the treatment and/or prevention of amyotrophic lateral sclerosis (ALS) may be any vector of the invention as defined above.

The host cell for use as a medicament in the treatment and/or prevention of amyotrophic lateral sclerosis (ALS) may be any host cell of the invention as defined above.

In the present, the terms “treatment,” “treat,” “treated” or “treating” refer to prophylaxis and/or therapy, particularly wherein the object is to prevent or slow down (lessen) an undesired physiological change or disorder, such as the development and/or progression of muscular disorders and/or inactivation and/or destruction of motoneurons. Beneficial or desired clinical results include, but are not limited to, alleviation of symptoms, diminishment of the extent of disease, stabilized (i.e., not worsening) state of disease, delay or slowing of disease progression, amelioration or palliation of the disease state, and remission (whether partial or total), whether detectable or undetectable. “Treatment” can also mean prolonging survival and/or increased quality of life as compared to expected survival and/or quality of life if not receiving treatment. Advantageously, treatment may include any of the following: decrease of alteration of motoneurons, for example inactivation and/or degradation of motoneurons, improvement of muscle strength, improvement of muscle size, decrease of alteration of walking, speaking and/or breathing capacities, decrease of fasciculations and/or cramps and/or improvement of walking, speaking, heating and/or breathing capacities, weight loss, decrease of upper motor neurones symptoms such as spasticity, hyperreflexia, emotional labidity.

A “subject” includes a mammal, e.g., a human, including a mammal in need of treatment for a disease or disorder, such as a mammal having been diagnosed with having a disease or disorder or determined to be at risk of developing a disease or disorder.

Another object of the present invention is a pharmaceutical composition comprising a nucleic acid and/or a vector and/or a host cell of the present invention.

The nucleic acid in the pharmaceutical composition may be any nucleic acid of the invention as defined above.

The vector in the pharmaceutical composition may be any vector of the invention as defined above.

The host cell in the pharmaceutical composition may be any host cell of the invention as defined above.

The pharmaceutical composition may be in any form that can be administered to a human or an animal.

Administration may be carried out directly, i.e. pure or substantially pure, or after mixing of the nucleic acid and/or a vector and/or a host cell of the present invention with a pharmaceutically acceptable carrier and/or medium. According to the present invention, the pharmaceutical composition may be a syrup or an injectable solution. According to the present invention, the pharmaceutical composition may be a pharmaceutical composition for oral administration selected from the group comprising a liquid formulation, an oral effervescent dosage form, an oral powder, a multiparticule system, an orodispersible dosage form. For example, when the pharmaceutical composition is for oral administration, it may be in the form of a liquid formulation selected from the group comprising a solution, a syrup, a suspension, an emulsion and oral drops. When the pharmaceutical composition is in the form of an oral effervescent dosage form, it may be in a form selected from the group comprising tablets, granules, and powders. When the pharmaceutical composition is the form of an oral powder or a multiparticulate system, it may be in a form selected from the group comprising beads, granules, mini tablets and micro granules. When the pharmaceutical composition is the form of an orodispersible dosage form, it may be in a form selected from the group comprising orodispersible tablets, lyophilized wafers, thin films, a chewable tablet, a tablet and a capsule, a medical chewing gum. According to the present invention, the pharmaceutical composition may be for buccal and sublingual routes, for example selected from the group comprising buccal or sublingual tablets, muco adhesive preparation, lozenges, oro-mucosal drops and sprays. According to the present invention, the pharmaceutical composition may be for topical-transdermal administration, for example selected from the group comprising ointments, cream, gel, lotion, patch and foam.

According to the present invention, the pharmaceutical composition may be for nasal administration, for example selected from the group comprising nasal drops, nasal spray, nasal powder. According to the present invention, the pharmaceutical composition may be for rectal administration, for example suppository or hard gelatin capsule. According to the present invention, the pharmaceutical composition may be for parenteral administration, for example subcutaneous, intramuscular, intravenous administration. The skilled person in the art understands clearly that the term “form” as used herein refers to the pharmaceutical formulation for its practical use.

The pharmaceutically acceptable carrier may be any know pharmaceutically carrier used for the administration of oligonucleotide/vector and or host cell to a human or to an animal, depending on the subject. For example, pharmaceutically acceptable carrier, diluent or excipient includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent or emulsifier. For example, this carrier may be selected from the group as described in Krützfeldt J, Rajewsky N, Braich R, Rajeev K G, Tuschl T, Manoharan M, Stoffel M. Nature. 2005 Dec. 1; 438(7068):685-9. Epub 2005 Oct. 30 [7].

The form of the pharmaceutical composition may be selected with regards to the human or animal to be treated.

In another aspect, the present invention provides a method of treating a subject suffering from amyotrophic lateral sclerosis (ALS), in particular juvenile amyotrophic lateral sclerosis (ALS). This method may comprise the step of administering to said subject a nucleic acid and/or vector and/or host cell of the invention.

Nucleic acid and/or vector and/or host cell of the invention, as well as usable formulations are as defined above. The administration can be made by using any pharmaceutical way known by the skilled person and useful to administrate nucleic acid and/or vector and/or host cell. Examples of administrable forms of medicament are provided above.

Other features and advantages will become further apparent to those skilled in the art upon reading the examples below, given by way of non-limiting illustration, with reference to the appended figures.

### BRIEF DESCRIPTION OF THE DRAWING

FIG. 1A is a scheme of the breeding strategy, in the scheme Fus +/+ means mice comprising wild type copy of the Fus gene, Fus ΔNLS/+ means mice comprising a copy of the Fus gene without the nuclear localization sequence (ΔNLS) and a wt copy of the Fus gene, hFUS means mice expressing a copy of the human wild type FUS gene. FIG. 1 B is a photography of a Western blot of Fus +/+ mice, Fus ΔNLS/+ mice, Fus ΔNLS/ΔNLS mice expressing or not a copy of the human wild type FUS gene (hFUS). In this photography, ΔNLS shows the migration line of the ΔNLS protein, wt shows the migration line of the wild type mouse Fus protein and hFUS the migration line of the wild type human Fus protein. FIG. 1 C is a diagram representing the Kaplan Meier survival curve of the different mice genotypes: Fus +/+ mice, Fus ΔNLS/+ and Fus ΔNLS/+; hFUS, the ordinate represents the percentage survival and the abscissa the time in days. FIG. 1 D is a diagram representing the Kaplan Meier survival curve of the different mice genotypes: Fus +/+ mice, Fus ΔNLS/ΔNLS and Fus ΔNLS/ΔNLS; hFUS, the ordinate represents the percentage survival and the abscissa the time in days.

FIG. 2 represents diagram comprising curves representing the age-dependent changes in the mean hanging time (s) (FIG. 2 A) and holding impulse (N s) (FIG. 2 B) in the four-limb wire inverted grid test with Fus +/+, and Fus ΔNLS/+ mice with or without hFUS transgene.

FIG. 3 represents the analysis of cellular localization of FUS protein. FIG. 3 A represents photography of immunoblot analysis of FUS protein subcellular localization in cortex of Fus +/+ (+/+) and Fus ΔNLS/+ (Δ/+) mice with (hFUS) or without hFUS (No Tg) transgene and of Fus ΔNLS/ΔNLS (Δ/Δ) mice with hFUS transgene at 1 month of age. Representative results using different antibodies targeting the N-terminal part (N-ter) of FUS (N-ter FUS), the C-terminal (C-ter) NLS FUS (C-Ter FUS), mouse FUS or human FUS. SOD1 and HDAC1 are used as loading controls for cytoplasmic and nuclear protein extracts fractions, respectively. FIG. 3 B represents histograms representing the quantification of N-ter FUS, C-ter FUS, mouse FUS and human FUS protein levels in cytoplasmic and nuclear fractions in Fus +/+ (+/+) and Fus ΔNLS/+ (Δ/+) mice with (hFUS) or without hFUS (No Tg) transgene and of Fus ΔNLS/ΔNLS (α/α) mice with hFUS transgene, in the figure * means p<0.05, *** means p<0.001 vs Fus+/+ mice, # means p<0.05 and ### means p<0.001 vs indicated mice genotype by ANOVA followed by Tukey. FIG. 3 C represents photographies of US immunohistochemistry in the spinal cord ventral horn at 22 months of age of in Fus +/+ (+/+) and Fus ΔNLS/+ (Δ/+) mice with (hFUS) or without hFUS (No Tg) transgene and of Fus ΔNLS/ΔNLS (α/α) mice with hFUS transgene. FIG. 3 D represents photographies of double immunostaining of the motoneuronal marker ChAT and FUS (N-terminal part) in the spinal cord ventral horn of mice at 22 months of age using anti-FUS and anti-ChAT antibodies.

FIG. 4 represents the analysis of accumulation of cytoplasmic asymmetrically demethylated (ADMA) FUS and the quantification of ADMA-FUS protein levels in cytoplasmic and nuclear fractions. FIG. 4A represents photographies of immunoblot analysis of asymmetrically arginine dimethylated FUS on cytoplasmic and nuclear fractions of cortex of Fus +/+ (+/+) and Fus ΔNLS/+ (Δ/+) mice with or without hFUS transgene and of Fus ΔNLS/ΔNLS mice with hFUS transgene at 1 month of age using an antibody recognizing asymmetrically arginine dimethylated FUS (ADMA-FUS), anti-HDAC1 antibody is used as a loading control for nuclear fractions and anti-SOD1 antibody is used as a loading control for nuclear fractions for cytoplasmic fractions. FIG. 4B represents histograms corresponding to the Quantification of ADMA-FUS protein levels in cytoplasmic and nuclear fractions of Fus +/+ (+/+) and Fus ΔNLS/+ (Δ/+) mice with or without hFUS transgene and of Fus ΔNLS/ΔNLS mice with hFUS transgene. In the figure ** means p<0.01 vs Fus+/+, # means p<0.05 vs indicated genotype by ANOVA analysis followed by Tukey. FIG. 4C represents represents photographies of immunostaining of with motoneuronal marker ChAT (red) and/or ADMA-FUS (C, green) in the spinal cord ventral horn of Fus +/+ (+/+) and Fus ΔNLS/+ (Δ/+) mice with or without hFUS transgene and of Fus ΔNLS/ΔNLS mice with hFUS transgene. FIG. 4C represents represents photographies of immunostaining of with motoneuronal marker ChAT (red) and/or ubiquitin (D, →) in the spinal cord ventral horn of Fus +/+ (+/+) and Fus ΔNLS/+ (Δ/+) mice with or without hFUS transgene and of Fus ΔNLS/ΔNLS mice with hFUS transgene.

FIG. 5 represents histograms representing gene expression in spinal cord determined with RT-qPCR. FIG. 5A are histograms representing the results of expression of endogenous mouse Fus mRNA (left), human FUS transgene (middle) and endogenous Fus mRNA deleted of exon 7 (right) in spinal cord at 1 month of age. FIG. 5B are histograms representing the results of expression of endogenous mouse Fus mRNA (left), human FUS transgene (middle) and endogenous Fus mRNA deleted of exon 7 (right) in spinal cord at 22 month of age.

FIG. 6 represents diagram comprising curves representing the age-dependent changes in the mean hanging time (s) (FIG. 6 A) and holding impulse (N s) (FIG. 6 B) in the four-limb wire inverted grid test with Fus +/+, Fus ΔNLS/+ mice with or without hFUS transgene and of Fus ΔNLS/ΔNLS mice with hFUS transgene.

FIG. 7 represents histograms representing gene expression in frontal cortex determined with RT-qPCR. FIG. 7A are histograms representing the results of expression of endogenous mouse Fus mRNA (left), human FUS transgene (middle) and endogenous Fus mRNA deleted of exon 7 (right) in spinal cord at 1 month of age. FIG. 7B are histograms representing the results of expression of endogenous mouse Fus mRNA (left), human FUS transgene (middle) and endogenous Fus mRNA deleted of exon 7 (right) in spinal cord at 22 month of age.

FIG. 8 represents diagrams corresponding to the level of expression of human FUS transgene (ordinate) compared to the level of endogenous Fus mRNA deleted of exon 7 in Fus ΔNLS/+ mice with hFUS mice 1 month or 22 months aged in spinal cord (FIG. 8A) or Frontal cortex (FIG. 8B).

FIG. 9 is a schematic representation of plasmid vector pMX.

FIG. 10 shows that insertion of intron 6 or intron 7 allows FUS autoregulation in transfected HEK293 cells. A: DNA constructs used: Myc-exon is an expression plasmid encoding uninterrupted human FUS ORF (exons 1 to 15: E1-15), N-terminally tagged by a Myc epitope; HA-i6 is an expression plasmid encoding the human FUS ORF, N-terminally tagged with an HA epitope and interrupted by human intron 6 between exon 6 (exons 1 to 6: E1-6) and exon 7 (exons 7 to 15: E7-15); HA-i7 is an expression plasmid encoding the human FUS ORF, N-terminally tagged with an HA epitope and interrupted by human intron 7 between exon 7 (exons 1 to 7: E1-7) and exon 8 (exons 8 to 15: E8-15). B: HEK293 cells transfected with an empty plasmid or Myc-exon, HA-i6 and HA-i7 constructs, and harvested 24 h after transfection. Representative immunoblot for FUS protein (@FUS), HA epitope (@HA) and Myc epitope (@Myc). Protein loading is shown as total protein strain of the corresponding western blot to demonstrate equal loading. The first well shows molecular weight marker. All three transgenic proteins are expressed at high levels. While total FUS protein levels are increased in Myc exon transfected cells, there is no change of FUS levels upon transfection of HA-i6 and HA-i7 constructs.

FIG. 11 shows that HA-i7 induces endogenous FUS intron 6 retention after 48 h of transfection. A: Endogenous Fus mRNA retaining intron 6 in HEK293 cells transfected 24 h or 48 h with Myc-exon or HA-i7 vectors. B: endogenous Fus mRNA retaining intron 6 in HEK 293 cells transfected 24 h or 48 h with Myc-exon, HA-i6 vectors.

FIG. 12 shows that alternative HA plasmid constructs are translated at the expected molecular weight for FUS in HEK293 cells after 24 h and 48 h of transfection. A: DNA constructs used: HA-1, 2 and 7 are expression plasmids encoding uninterrupted human FUS ORF (exons 1 to 15: E1-15), N-terminally tagged by a HA epitope and interrupted by human RNA preserved motif in intron 6 between exon 6 and 7. HA-3 to 6 and HA-8 are expression plasmids encoding uninterrupted human FUS ORF (exons 1 to 15: E1-15), N-terminally tagged by a HA epitope and interrupted by human RNA preserved motif in intron 7 between exon 7 and 8. HA-9 to 12 are expression plasmids encoding uninterrupted human FUS ORF (exons 1 to 15: E1-15), N-terminally tagged by a HA epitope and interrupted by human RNA preserved motif in introns 6 and 7. RNA preserved motifs are based on Zhou et al. (“ALS-associated FUS mutations result in compromised FUS alternative splicing and autoregulation”. October 2013, Volume 9, Issue 10, e1003895) showing a mapping of CLIP tags in the FUS intron6-exon7-intron7 region. B: HA protein levels in HEK293 cells transfected 3 independent times during 24 h and 48 h with empty Myc-exon and HA-1 to 12 vectors.

### EQUIVALENTS

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### EXEMPLIFICATION

The present invention and its applications can be understood further by the examples that illustrate some of the embodiments by which the inventive product and medical use may be reduced to practice. It will be appreciated, however, that these examples do not limit the invention. Variations of the invention, now known or further developed, are considered to fall within the scope of the present invention as described herein and as hereinafter claimed.

### EXAMPLES

### Example 1

### Nucleic Acid and Vector Comprising said Nucleic Acid

The nucleic acid and recombinant vector are prepared and obtained as disclosed in GeneArt Gene Synthesis products and services form thermofisher scientific.

The nucleic acid used were SEQ ID NO 1 to 20. The nucleic acid were prepared and included if necessary the sequences for the restriction site Not1 (GCGGCCGC) and Xba1 (TCTAGA) for litigation with the plasmid. The prepared nucleic acid is 5 μg lyophilized plasmid DNA

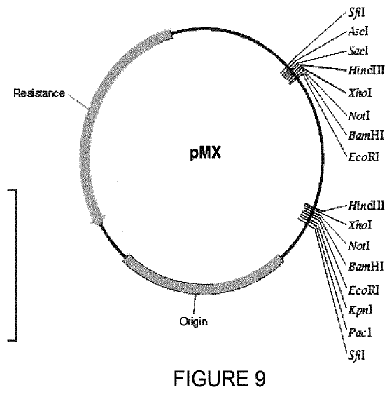

The vector used is a plamid vector pMX which is represented in FIG. 9 and described in Retrovirus-mediated gene transfer and expression cloning: powerful tools in functional genomics. Exp Hematol. 2003 November; 31(11):1007-14. Kitamura T, Koshino Y, Shibata F, Oki T, Nakajima H, Nosaka T, Kumagai H. Retroviral vector designed for expression cloning and efficient gene transfer. The pMX vector harbors 5 long terminal repeat (LTR) and the extended packaging signal derived from MFG followed by a multi-cloning site (MSC) suitable for cDNA library construction and 3 LTR of MMLV.

10 ng of DNA were incubated 30 min on ice with 50 μl of competent cells (Invitrogen, 18265-017). An heat shock was performed 20 seconds at 42° C. in water bath. Then tubes were places 2 min on ice then incubate with medium at 37° C. for 1 hour at 225 rpm. Thereafter an agar culture with antibiotic was performed overnight at 37° C. A single colony was inoculated in liquid culture with antibiotic overnight at 37° C., 225 rpm then plasmid midi kit was performed using manufacturer's recommendation (Qiagen, 12643).

### Example 2

### In Vivo Treatment of a Mouse Model of FUS-ALS with a Nucleic Acid Encoding Human FUS Protein Intron 6 and/or a Sequence Intron 7

### Materials and Methods

### Mouse Models and Genotyping

Mouse experiments were approved by local ethical committee from Strasbourg University (CREMEAS) under reference number 2016111716439395.

The vector used is a BAC comprising the nucleic acid sequence coding for the human FUS of SEQ ID NO 10. Transgenic mice were generated as described in Scekic-Zahirovic et al. 2016 and in Scekic-Zahirovic 2017 [11], were bred in Charles River animal facility and housed in the Faculty of medicine from Strasbourg University with 12/12 hours of light/dark cycle (light on at 7:00 am) under constant conditions (21±1° C.; 60% humidity) and with unrestricted access to food and water.

Mice were weaned and genotyped at 21 days by PCR from tail biopsy. The following primer sequences were used to genotype mice:

hTLS FUS-For:
(SEQ ID NO 21)
GAATTCGTGGACCAGGAAGGTC
hTLS FUS-Rev:
(SEQ ID NO 22)
CACGTGTGAACTCACCGGAGTCA
FUS-For:
(SEQ ID NO 23)
GATTTGAAGTGGGTAGATAGTGCAGG
FUS-Rev:
(SEQ ID NO 24)
CCT-TTC-CAC-ACT-TTA-GTT-TAG-TCA-CAG

Heterozygous Fus knock-in mice, lacking the PY-NLS, were crossed with mice expressing human wild type FUS from a complete, autoregulatory competent, human gene to obtain following genotypes: Fus+/+, FusΔNLS/+, FusΔNLS/ΔNLS, Fus+/+: hFUS, Fust NLS/+: hFUS, FusΔNLS/ΔNLS: hFUS. The genetic background of all mice used in this study is C57BI6/J.

### Mouse Behavior

Survival

Survival was studied during the first hours after birth and dead new born mice were genotype. Other mice were genotyped at 21 days and followed weakly until death or killed using ketamine-xylazine when they reach the following endpoints: auto-mutilation, weight loss greater than 10% of the initial weight and when they could not turn around again within 10 seconds after being laid on their side.

Inverted Grid

Motor performance were performed weakly as described previously (Jelena Acta Neuropathol 2017 [8]). The wire grid hanging time (or “hang time”) was defined as the amount of time that it takes the mouse to fall down from the inverted grid and was measured visually with a stop watch. The procedure was repeated 3 times during 5 min. The holding impulse corresponds to hanging time normalized with mouse weight and gravitational force.

### Histological Techniques

Mice were anesthetized with intraperitoneal injection of 100 mg/kg ketamine chlorhydrate and 5 mg/kg xylazine then perfused with PFA 4%. After dissection, spinal cord was included in agar 4% and serial cuts of 40 μm thick were made with vibratome.

Peroxydase Immunohistochemistry

For peroxidase immunohistochemistry, endogenous peroxidases were inactivated 10 min with H₂O₂3% then slides were washed with PBS 1× and incubated overnight with rabbit anti-FUS antibody (ProteinTech, 11570-1-AP, 1:100) at room temperature. After rinsing in PBS, anti-rabbit biotinylated (Jackson, 711-067-003, 1/500) was incubated 2 h at room temperature. The staining was revealed using the ABC kit (Vectastain ABC kit, PK-6100, Vector Laboratories Inc.) during 1 h. After 3 washes with Phosphate Buffer Salin (PBS), slides were rinse in water and mounted in DPX (Sigma, O6522).

Immunofluorescence

Sections were incubated in blocking solution (8% Goat serum, 0.3% Bovine Serum Albumin, 0.3% Triton, PBS-0.02% Thimerosal) at room temperature, i.e. 25° C., then incubate overnight at 4° C. in primary antibody:rabbit anti-FUS antibody (ProteinTech, 11570-1-AP, 1:100), goat anti-ChAT (Milliport, AB144P, 1/50), rat anti-ADMA (Home Made, Germany, 1/100) or rabbit anti-Ubi (Abcam, ab179434, 1/100). After 3 rinses in PBS, sections were incubated 2 h at room temperature i.e 25° C. with Hoechst (Sigma, B2261, 1/50.000) and secondary antibody: Alexa-488-conjugated anti-rabbit secondary antibody (Jackson, 711-547-003, 1/500) or Alexa-594-conjugated anti-goat secondary antibody (Molecular Probes, A 11058, 1/500). Finally sections were subsequently washed with PBS (3×10 min) and mounted in DPX (Sigma, O6522).

Electron Microscopy

Mice were anesthetized with intraperitoneal injection of 100 mg/kg ketamine chlorhydrate and 5 mg/kg xylazine and transcardiacally perfused with glutaraldehyde (2.5% in 0.1M cacodylate buffer at pH 7.4). Brains were dissected and immersed in the same fixative, i.e. glutaraldehyde, overnight, i.e. 12 hours. After 3 rinses in Cacodylate buffer (EMS, 11650), muscles were post fixed in 0.5% osmium and 0.8% potassium ferrocyanide in Cacodylate buffer 1 h at room temperature. Finally, tissues were dehydrated in graded ethanol series (25%, 50%, 70%, 95%, 100%), and embedded in Embed 812 (EMS, 13940). The ultrathin sections (50 nm) were cut with an ultramicrotome (Leica, EM UC7), counterstained with uranyl acetate (1% (w/v) in 50% ethanol) and observed with a Hitachi 7500 transmission electron microscope (Hitachi High Technologies Corporation, Tokyo, Japan) equipped with an AMT Hamamatsu digital camera (Hamamatsu Photonics, Hamamatsu City, Japan).

Tissue Fractionation and Western Blotting

Tissues were washed in Phosphate Buffer Saline (PBS) 1× and lysed in Syn-PER Synaptic Protein Extraction (Thermo Scientific, 87793) according to the manufacturer's instructions. Protein extract were dosed by BCA Assay (Interchim, UP95424A, UP95425A). Thereafter proteins were denatured and SDS page were performed with 10 (for cytoplasmic proteins) and 30 μg of protein (for nuclear proteins) on criterion TGX stain free gel 4-20% (Biorad, 5678094). Proteins were blotted on nitrocellulose membrane using semi-dry Transblot Turbo system (BioRad, France) and blocked with 10% non-fat milk during 1 h. Primary antibodies (Rabbit anti-hFUS (Home Made, #14080, 1/2000), Rabbit anti-mFUS (Home Made, #14082, 1/4000), Rat anti-FUS ADMA (Home made, Germany, 1/500), Rabbit anti-FUS 293 (Bethyl, A-300-293A, 1/2000), Rabbit anti-FUS 294 (Bethyl, A300-294A, 1/2000), Sheep anti-SOD1 (Calbiochem, 574597, 1/1000), Rabbit anti-HDAC1 (Bethyl, A300-713A, 1/1000) were incubated overnight, i.e 12 hours, at 4° C. in 3% non-fat milk. Washing were proceeded with washing buffer (Tris pH 7.4 1 M, NaCl 5M, Tween 20 100%) and secondary antibody (anti-rabbit HRP (PARIS, BI2407, 1/5000), anti-sheep HRP (Jackson, 713-035-147, 1/5000) were incubated 1 h 30 at room temperature i.e 25° C. After successive washes, i.e. 3 washes, with PBS1× proteins were visualized with chemiluminescence using ECL Lumina Forte (Millipore, France) and chemiluminescence detector (Bio-Rad, France). Total proteins were detected with stain free gel capacity (Biorad, 5678094) and used to normalized.

Antibodies used were the followings:
Rabbit anti-hFUS (Home Made, #14080, 1/2000),
Rabbit anti-mFUS (Home Made, #14082, 1/4000)
Rat anti-FUS ADMA (Home made, Germany, 1/500)
Rabbit anti-FUS 293 (Bethyl, A-300-293A, 1/2000)
Rabbit anti-FUS 294 (Bethyl, A300-294A, 1/2000)
Sheep anti-SOD1 (Calbiochem, 574597, 1/1000)
Rabbit anti-HDAC1 (Bethyl, A300-713A, 1/1000)
Anti-rabbit HRP (PARIS, BI2407, 1/5000)
Anti-sheep HRP (Jackson, 713-035-147, 1/5000)

### RNA Extraction and RT-qPCR

Total RNA was extracted from spinal cord and frontal cortex using Tissue Lyser (Qiagen) in 100 μl of TRIzol® reagent (Life Technologies).

1 μg of RNA was reverse transcribed with iScript™ reverse transcription using manufacturer conditions (Biorad, 1708841). Quantitative polymerase chain reaction was performed using Sso Advanced Universal SYBR Green Supermix (Bio-Rad) and quantified with Bio-Rad software using manufacturer conditions. Gene expression was normalized by calculating a normalization factor using actin, TBP and pol2 genes according to GeNorm software (Vandesompe et al 2002) Primer sequences were as follows:

Actin:
F-
(SEQ ID NO 25)
CCACCAGTTCGCCATGGAT,
R-
(SEQ ID NO 26)
GGCTTTGCACATGCCGGAG
TBP:
F-
(SEQ ID NO 27)
CCAATGACTCCTATGACCCCTA,
R-
(SEQ ID NO 28)
CAGCCAAGATTCACGGTAGAT
Pol2:
F-
(SEQ ID NO 29)
GCTGGGAGACATAGACCA,
R-
(SEQ ID NO 30)
TTACTCCCCTGCATGGTCTC
hFUS:
F-
(SEQ ID NO 31)
GCCAGAACACAGGCTATGGA,
R-
(SEQ ID NO 32)
CGATTGGGAGCTCTGGCTAC
T-FUS:
F-
(SEQ ID NO 33)
TTATGGACAGACCCAAAAACACA
R-
(SEQ ID NO 34)
TGCTGCCCATAAGAAGATTG
FUS ANLS:
F-
(SEQ ID NO 35)
GCAAGATGGACTCCTAGTGTTAAT,
R-
(SEQ ID NO 36)
ACCTCTACAAATGTGGTATGGC
Fus_exon6-8:
F-
(SEQ ID NO 37)
CGGCATGGGGTCCTCGG,
R-
(SEQ ID NO 38)
CCTAGGCCTTGCACGAAGAT

### Statistics

All results from analysis are presented as mean±standard error of the mean (SEM) and differences were considered significant when p<0.05. Significance is presented as follows: * p<0.05, ** p<0.01, and *** p<0.001. For comparison of two groups, two-tailed unpaired Student's t—test was used in combination with F-test to confirm that the variances between groups were not significantly different. Comparison for more than two groups was performed using one-way ANOVA and Tukey or Bonferroni's multiple comparison post hoc test. Data were analyzed by using the GrapPad Prism version 6.0.

### Results

### Human Wild Type FUS Transgene Rescues Perinatal Letality in Homozygous FusΔNLS Mice

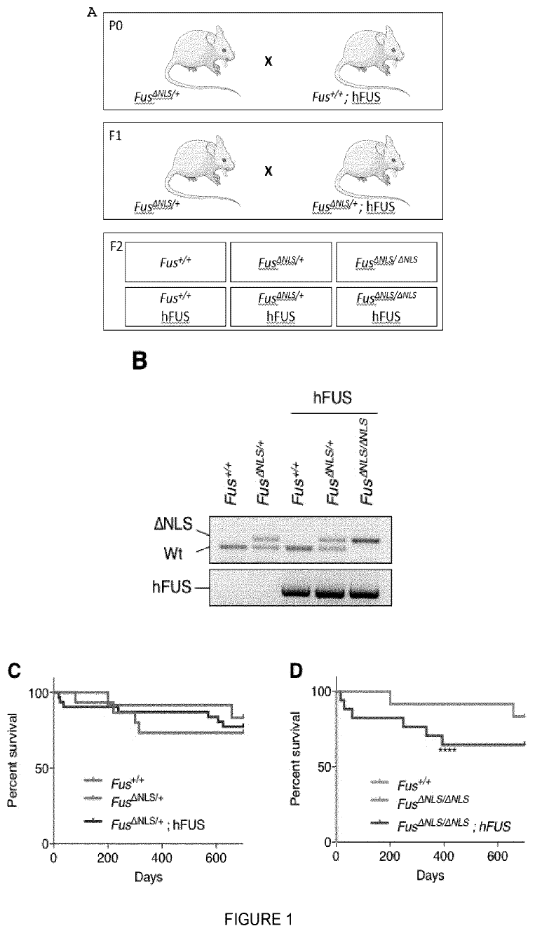

Human wild type FUS transgenic mice (hFUS mice) expressing wild type FUS from a BAC have been recently generated and characterized (Lopez-Erauskin J, Tadokoro T, Baughn M W, et al. ALS/FTD-Linked Mutation in FUS Suppresses Intra-axonal Protein Synthesis and Drives Disease Without Nuclear Loss-of-Function of FUS. Neuron 2018; 100(4): 816-30 e7 [9]). These mice express slightly increased FUS levels, mostly of human origin, and do not show ALS-related phenotypes contrary to the human mutant FUS transgenic lines generated and characterized in parallel (Lopez-Erauskin J, Tadokoro T, Baughn M W, et al. ALS/FTD-Linked Mutation in FUS Suppresses Intra-axonal Protein Synthesis and Drives Disease Without Nuclear Loss-of-Function of FUS. Neuron 2018; 100(4): 816-30 e7 [9]). hFUS mice were crossed with FusΔNLS mice (Scekic-Zahirovic J, Sendscheid O, El Oussini H, et al. Toxic gain of function from mutant FUS protein is crucial to trigger cell autonomous motor neuron loss. EMBO J 2016; 35(10): 1077-97 [10]), that we previously showed to develop mild, late onset motor neuron disease as heterozygous (Scekic-Zahirovic J, Oussini H E, Mersmann S, et al. Motor neuron intrinsic and extrinsic mechanisms contribute to the pathogenesis of FUS-associated amyotrophic lateral sclerosis. Acta Neuropathol 2017; 133(6): 887-906 [11]) while homozygous FusΔNLS mice die in the perinatal period (Scekic-Zahirovic J, Sendscheid O, El Oussini H, et al. Toxic gain of function from mutant FUS protein is crucial to trigger cell autonomous motor neuron loss. EMBO J 2016; 35(10): 1077-97 [10]). After two breeding steps, genotypes of interest, including heterozygous and homozygous FusΔNLS mice with or without the hFUS transgene were obtained (FIG. 1A). Of note, all mice were in C57BI/6 genetic background, and only mice from the F2 generation were analyzed here, thereby avoiding most of the possible confounding effects of genetic background heterogeneity. Mice were genotyped at 1 month of age or at death if occurring before 1 month of age. As demonstrate in FIG. 1, D FusΔNLS/ΔNLS mice without hFUS died within the first hours after birth and we did not observe mice with this genotype at 1 month of age (FIG. 1C). Contrastingly, the addition of the human wild type FUS transgene allowed the survival of most homozygous FusΔNLS mice until adulthood (FIGS. 1B, D). A slight but non statically significant increased mortality of all FusΔNLS groups as compared to wild type animals was observed (FIG. 1D).

### Human Wild Type FUS Transgene Rescues Late Onset Muscle Weakness in Heterozygous FusΔNLS Mice

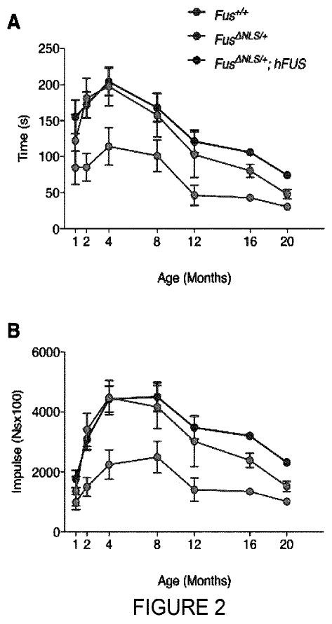

The mice used were Fus +/+, Fus ΔNLS/+, and Fus ΔNLS/+ expressing H FUS mice. The number of mice used were N=10-28 per group. As shown on FIG. 2, FusΔNLS/+ mice develop mild, late onset, muscle weakness, that can be easily followed using inverted grid test. Using this test, the deficit in FusΔNLS/+ mice occurred was observed after 2 months of age and was stable with age (FIGS. 2A-B). Contrastingly, FusΔNLS/+ mice with a human wild type FUS transgene were undistinguishable from wild type littermates in this test, suggesting that the wild type FUS transgene was sufficient to rescue the neuromuscular phenotype (FIGS. 2A-B).

### Human Wild Type FUS Transgene Rescues Cytoplasmic Mislocalization of the FUS Protein in FusΔNLS Mice

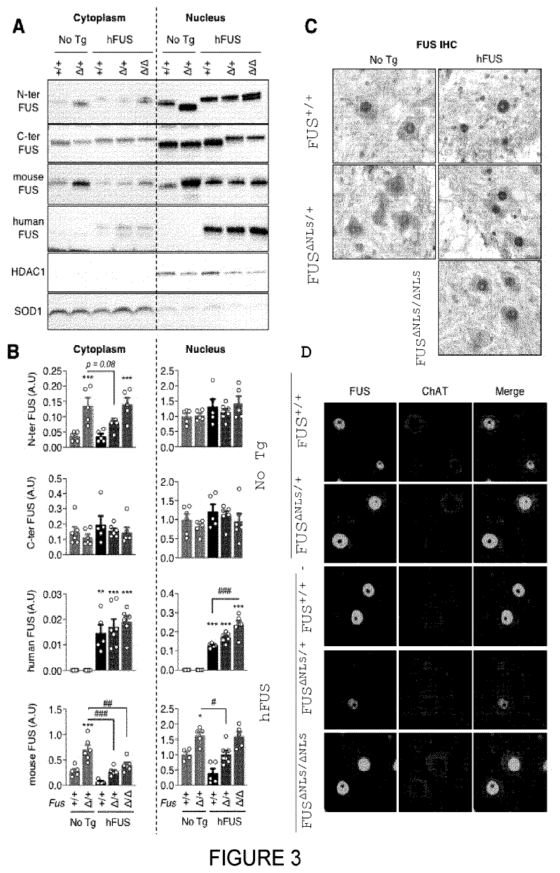

The effect of the expression of human wild type FUS transgene on the subcellular localization of FUS in FusΔNLS mice was studied. The results obtained are represented on FIG. 3. As demonstrated on FIGS. 3A and 3B, the cytoplasmic fractions of cerebral cortex of FusΔNLS/+ mice displayed higher levels of FUS than corresponding wild type littermate fractions as assessed using western blotting. This was not observed when an antibody targeting the NLS sequence, absent from the FUSΔNLS protein, was used, demonstrating that this increase is related to the mislocalization of the mutant protein. Importantly, mouse FUS, as identified using a mouse specific FUS antibody, was increased in cytoplasmic fractions of FusΔNLS/+ mice, and this was normalized by the human wild type FUS transgene (FIGS. 3A-B). Importantly, nuclear FUS levels were unchanged in all genotypes, irrespective of the presence of the FusΔNLS mutation or of the human wild type FUS transgene. Human FUS levels were increased in FusΔNLS/ΔNLS mice carrying a human FUS transgene, likely compensating for the loss of nuclear FUS of mouse origin. To further confirm this rescuing effect of the transgene, immunohistochemistry on spinal cord sections of 22 months old mice was performed. As shown in FIG. 3C, FusΔNLS/+ neurons showed lacked nuclear enrichment in FUS staining, and this was fully prevented by the human wild type FUS transgene, whether in FusΔNLS/+ mice or in FusΔNLS/ΔNLS mice. Double immunofluorescence using FUS and ChAT antibodies to unambiguously identify motor neurons further confirmed that the human wild type FUS transgene rescued FUS mislocalization in motor neurons as demonstrated and shown on FIG. 3D.

This results clearly demonstrate that products, for example nucleic acid and/or vectors, of the present invention allow in the same time and synergistically to compensate the genetic mutation by providing wild-type protein FUS, to down regulate the expression and the production of mutated protein FUS, and to regulate the expression and production of wild-type protein FUS.

### Human Wild Type FUS Transgene Rescues ALS-Related Pathology in Heterozygous FusΔNLS Mice

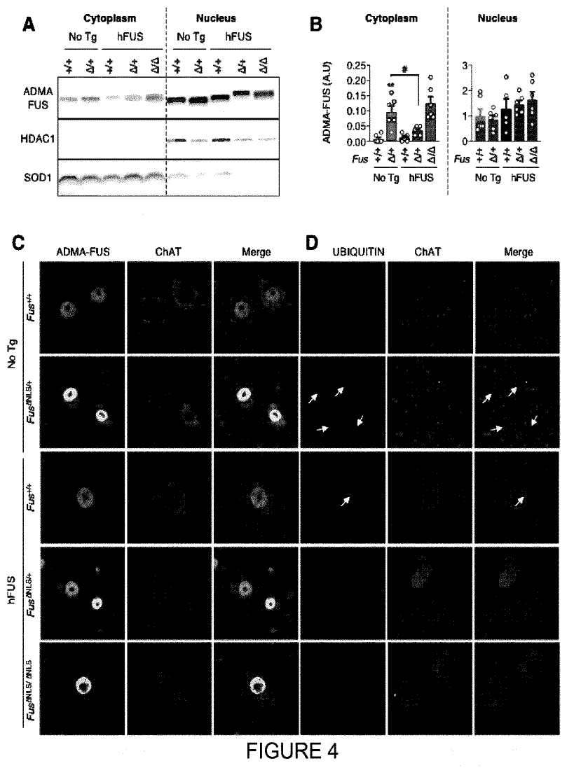

To further document the protective effect of the human wild type FUS transgene, pathological hallmarks developed by Fus +/+ (+/+) and Fus ΔNLS/+ (Δ/+) mice with (hFUS) or without hFUS (No Tg) transgene and of Fus ΔNLS/ΔNLS (α/α) mice with hFUS transgene mice was analysed. The number of mice used were N=4-8 per group. Accumulation of cytoplasmic asymmetrically demethylated (ADMA) FUS is typical of FUS-ALS, and has been previously observed in FusΔNLS/+ mice. Indeed, the large increase in ADMA-FUS in FusΔNLS/+ cytoplasmic fractions was largely prevented in FusΔNLS/+ mice with a wild type human FUS transgene (FIGS. 4A-B). This was however not the case in FusΔNLS/ΔNLS mice carrying a hFUS transgene that retained high levels of cytoplasmic methylated FUS. Consistently, while ADMA-FUS immunoreactivity is diffuse in motor neurons of FusΔNLS/+ mice, the human wild type FUS transgene led to more localized, perinuclear immunoreactivity in FusΔNLS/+ mice (FIG. 4C). Furthermore, the punctate ubiquitin pathology observed in FusΔNLS/+ motor neurons was also prevented by the human wild type FUS transgene (FIG. 4D). Thus, the human wild type FUS transgene was able to rescue typical hallmarks of ALS-FUS pathology in FusΔNLS/+ mice.

This results clearly demonstrate that products, for example nucleic acid and/or vectors, of the present invention allow synergistically to eliminate the cytoplasmic accumulation of mutated FUS, to produce wild-type protein FUS and also to regulate the production of the wild-type protein FUS. In addition this results clearly demonstrate that products, for example nucleic acid and/or vectors, of the present invention allow to decrease the expression of the mRNA of mutated FUS protein and to restore and correct the function of FUS protein.

### Human Wild Type FUS Transgene Reverts Overexpression of Endogenous Fus and Activates Exon 7 Skipping

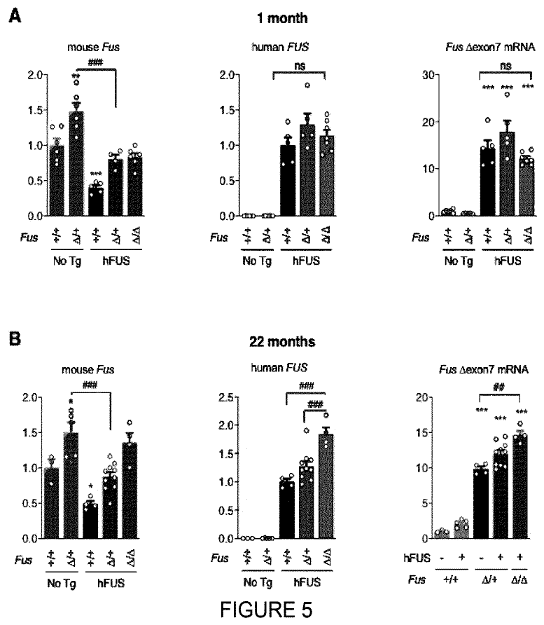

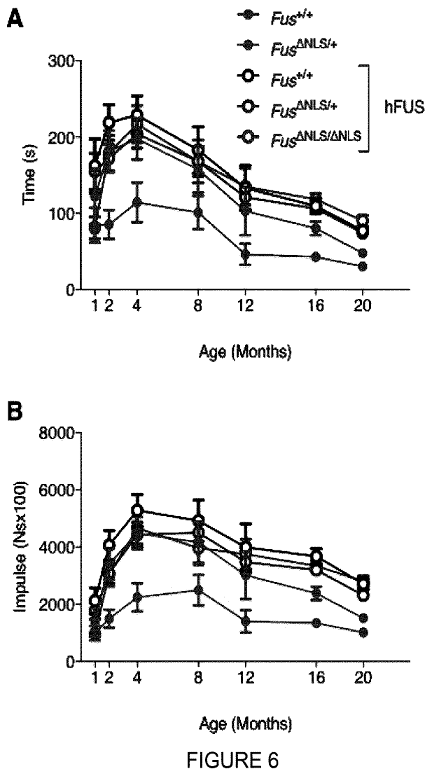

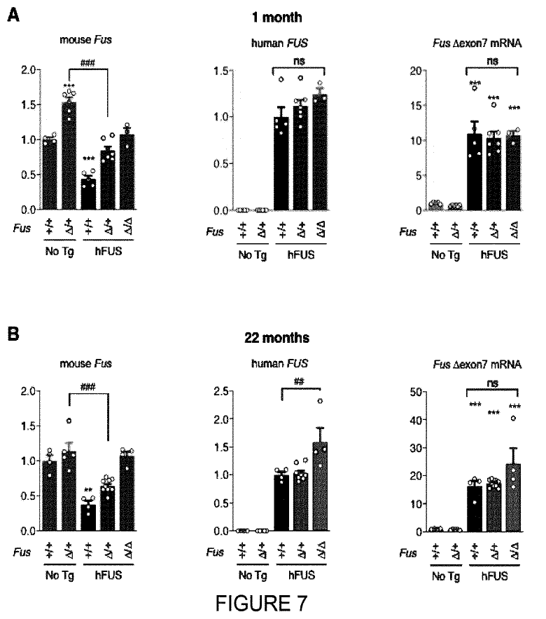

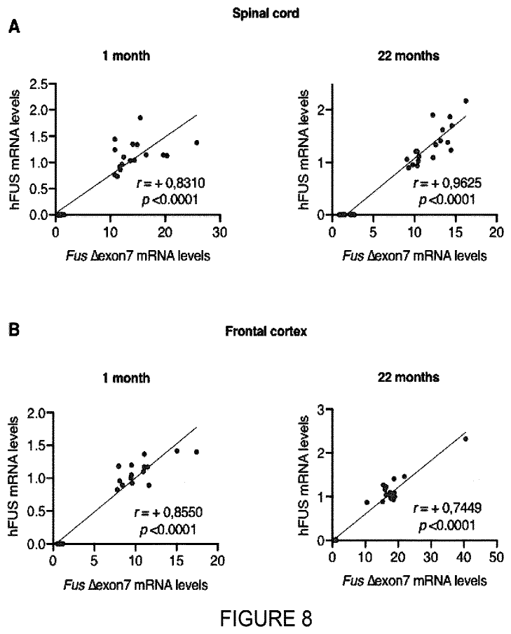

To further document the protective and/or therapeutic effect of the human wild type FUS transgene on the regulation of expression of endothelial FUS gene in Fus +/+ (+/+) and Fus ΔNLS/+ (Δ/+) mice with (hFUS) or without hFUS (No Tg) transgene and of Fus ΔNLS/ΔNLS (α/α) mice with hFUS transgene mice was analysed. Single human wild type FUS transgenic mice had lower expression of the endogenous Fus gene15 while FusΔNLS/+ mice displayed a slight but significant increase in Fus mRNA levels at both 1 and 22 months of age in spinal cord (FIGS. 5A-B) and frontal cortex (FIG. 7). This overexpression of the endogenous murine gene was fully corrected by the human wild type FUS transgene. Furthermore, the expression of the human FUS transgene was accompanied by strongly increased levels of the aberrantly spliced mRNA of Fus devoid of exon 7, substrate to mRNA decay (FIGS. 5A-B). Indeed, there was a strong correlation between hFUS expression and deletion of exon 7 in endogenous mouse Fus mRNA (Supplementary FIG. 3). Thus, the addition of the human wild type FUS transgene leads to decreased expression of the toxic protein through the activation of the autoregulatory loop, and subsequent alleviation of all the downstream consequences of the expression of cytoplasmically mislocalized FUS.

This example clearly demonstrates that products, for example nucleic acid and/or vectors, of the present invention allow to treat and cure amyotrophic lateral sclerosis (ALS), and in particular juvenile amyotrophic lateral sclerosis (ALS). In particular, this example clearly demonstrates that the present invention allow in the same time to decreased expression of the toxic protein, i.e mutated FUS, to produce a functional and homologous protein of human FUS protein, to activate of the autoregulatory loop for mutated FUS, to activate the autoregulatory loop for the expression of the nucleic acid of the invention allowing a wildtype expression of said nucleic acid and to eliminate the cytoplasmic and/or nucleus accumulation/mislocalization of the mutated FUS. Accordingly this example clearly demonstrates that surprisingly and unexpectedly, the present invention allow in the same time to “cancel” the biological default and to restore the wild-type biological mechanism.

### Example 3

### Effects of Insertion of Intron 6, 7 or Alternative FUS DNA Constructs in Mammalian Cells

### Insertion of Intron 6 or 7 Allows FUS Autoregulation in Transfected HEK293 Cells

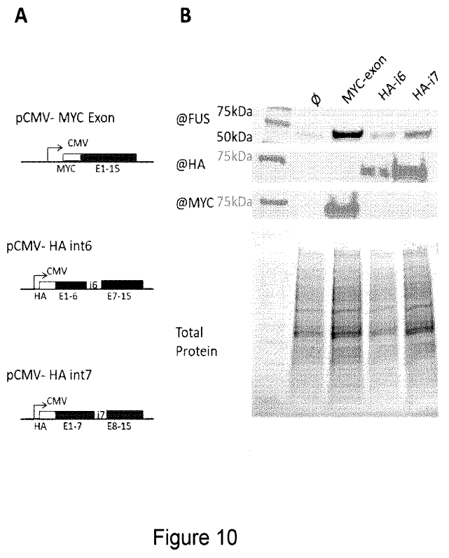

Intron 6 or intron 7 was inserted in the FUS ORF at their endogenous location in the pre-mRNA (between exons 6 and 7 for intron 6, and between exons 7 and 8 for intron 7). An N-terminal HA-tag was included to allow for the unambiguous detection of the transgenic protein (HA-i6 and HA-i7), and compared to a myc-tagged FUS expression plasmid (Myc exon) as a positive control (FIG. 10 A).

24 hours after transfection, we observed strong expression of an HA-immunoreactive protein, at the expected molecular weight for FUS for both HA-i6 and HA-i7 constructs (FIG. 10 B), showing that the insertion of the introns did not impair splicing and proper translation of the cDNA. Most importantly, while Myc-exon expression led to dramatic upregulation of FUS levels, HA-i6 and HA-i7 transfected cells showed normal levels of FUS, either 24 or 48 h after transfection (FIG. 10 B). Thus, insertion of intron 6 or intron 7 in the FUS ORF avoids overexpression observed upon expression of an uninterrupted cDNA, suggesting proper autoregulation.

HA-i6 Induces Endogenous FUS Intron 7 Retention after 48 h of Transfection

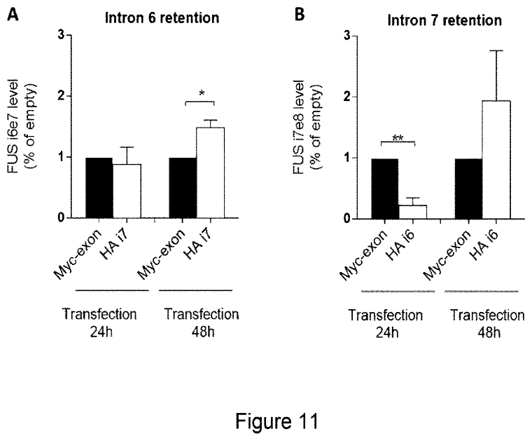

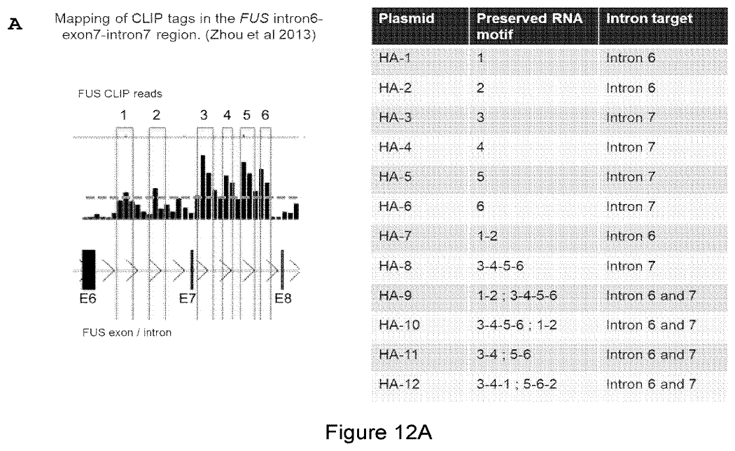

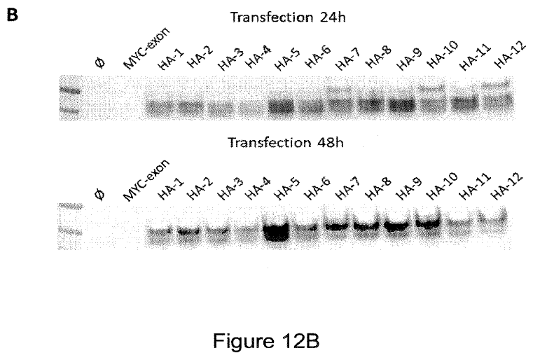

HEK293 cells were transfected with either Myc-exon or HA-i6, and measured retention of intron 7, that is not included in the HA-i6 construct. As shown in FIG. 11A, retention of endogenous intron 7 was identical in HA-i6 transfected cells as compared to Myc-exon 24 h after transfection. Then an increase of intron 7 retention level was observed 48 h after transfection in HA i6 transfected cells. Similarly, we observed increased levels of endogenous intron 6 retention in HEK293 cells transfected with HA-i7 as compared with Myc-exon transfected cells at 48 h after transfection (FIG. 11 B). These results provide proof of principle that HA-i6 and HA-i7 engage autoregulation of the endogenous mRNA for FUS.

### Alternative FUS DNA Constructs Allow Translation of HA-Tagged Proteins

Series of constructs were made, based on modifications of HA-i6 and HA-i7. All these constructs allowed the expression of an HA-immunoreactive band at the expected molecular weight for FUS in transfected HEK293 cells, especially for construct #9. Thus, engineering of the FUS gene can be performed to allow for shortening of the construct, while maintaining autoregulation and proper expression of the FUS protein.

### Methods

Cell Culture

HEK293 cells were purchased from ATCC (ATCC® CRL-1573™). Cells were cultured in Dulbecco's modified Eagle's medium containing 10% Foetal Bovine Serum (Fisher scientific, 11531831), 1% Penicilin-Streptomycin (Sigma, P4333) at 37° C. and in an incubator with 5% CO2. Culture medium was changed every two days and transfections were performed between 5-20 passages.

Transfection

C2C12 were cultured in 24 wells plate until 80% of confluency. Transfection was performed in differentiation medium with expression and reporter plasmids using TransIT-X2 (MIR6000, Myrus) according to the manufacturer's instructions. The nucleic acid were prepared and included if necessary the sequences for the restriction site Not1 (GCGGCCGC) and Xba1 (TCTAGA) for litigation with the plasmid. Expression vectors used for transfections were:
pCMV empty plasmid;
pCMV-Myc-FUS, expressing a N-terminal, myc-tagged human FUS without introns;
pCMV-HA-i6, expressing an HA-tagged human FUS with intron 6;
pCMV-HA-i7, expressing an HA-tagged human FUS with intron 7;
pCMV-HA-1, expressing an HA-tagged human FUS with a preserved RNA motif in intron 6 as shown in SEQ ID NO: 42. The construction was as followed: Not1-HA-Exon1-Exon2-Exon3-Exon4-Exon5-Exon6-motif1-Exon7-Exon8-Exon9-Exon10-Exon11-Exon12-Exon13-Exon14-Exon15-Xba1. The sequence of motif 1 of intron 6 corresponds to SEQ ID NO 54;
pCMV-HA-2, expressing an HA-tagged human FUS with a preserved RNA motif in intron 6 as shown in SEQ ID NO: 43. The construction was as followed: Not1-HA-E1-E2-E3-E4-E5-E6-motif2-E7-E8-E9-E10-E11-E12-E13-E14-E15-xba1. The sequence of motif 2 of intron 6 corresponds to SEQ ID NO 55;
pCMV-HA-3, expressing an HA-tagged human FUS with a preserved RNA motif in intron 7 as shown in SEQ ID NO: 44. The construction was as followed: Not1-HA-E1-E2-E3-E4-E5-E6-E7-motif3-E8-E9-E10-E11-E12-E13-E14-E15-xba1. The sequence of motif 3 of intron 7 corresponds to SEQ ID NO 56;
pCMV-HA-4, expressing an HA-tagged human FUS with a preserved RNA motif in intron 7 as shown in SEQ ID NO: 45. The construction was as followed: Not1-HA-E1-E2-E3-E4-E5-E6-E7-motif4-E8-E9-E10-E11-E12-E13-E14-E15-xba1. The sequence of motif 4 of intron 7 corresponds to SEQ ID NO 57;
pCMV-HA-5, expressing an HA-tagged human FUS with a preserved RNA motif in intron 7 as shown in SEQ ID NO: 46. The construction was as followed: Not1-HA-E1-E2-E3-E4-E5-E6-E7-motif5-E8-E9-E10-E11-E12-E13-E14-E15-xba1. The sequence of motif 5 of intron 7 corresponds to SEQ ID NO 58;
pCMV-HA-6, expressing an HA-tagged human FUS with a preserved RNA motif in intron 7 as shown in SEQ ID NO: 47. The construction was as followed: Not1-HA-E1-E2-E3-E4-E5-E6-E7-motif6-E8-E9-E10-E11-E12-E13-E14-E15-Xba1. The sequence of motif 6 of intron 7 corresponds to SEQ ID NO 59;
pCMV-HA-7, expressing an HA-tagged human FUS with 2 preserved RNA motifs in intron 6 and 4 preserved RNA motifs in intron 7 as shown in SEQ ID NO 48. The construction was as followed: Not1-HA-E1-E2-E3-E4-E5-E6-motif1-motif2-E7-motif3-motif4-motif5-motif6-E8-E9-E10-E11-E12-E13-E14-E15-Xba1;
pCMV-HA-8 (expressing an HA-tagged human FUS with 2 preversed RNA motifs in intron 6, as shown in SEQ ID NO 49. The construction was as followed: Not1-HA-E1-E2-E3-E4-E5-E6-motif1-motif2-E7-E8-E9-E10-E11-E12-E13-E14-E15-Xba1;
pCMV-HA-9, expressing an HA-tagged human FUS with 4 preversed RNA motifs in intron 7 as shown in SEQ ID NO 50. The construction was as followed: Not1-HA-E1-E2-E3-E4-E5-E6-E7-motif3-motif4-motif5-motif6-E8-E9-E10-E11-E12-E13-E14-E15-Xba1;
pCMV-HA-10, expressing an HA-tagged human FUS with 6 preserved RNA motifs in intron 6 and 7, as shown in SEQ ID NO 51. The construction was as followed: Not1-HA-E1-E2-E3-E4-E5-E6-motif1-motif2-E7-motif3-motif4-motif5-motif6-E8-E9-E10-E11-E12-E13-E14-E15-Xba1;
pCMV-HA-11, expressing an HA-tagged human FUS with 4 preserved RNA motifs in intron 6 and 7, as shown in SEQ ID NO 52. The construction was as followed: Not1-HA-E1-E2-E3-E4-E5-E6-motif3-motif4-E7-motif5-motif6-E8-E9-E10-E11-E12-E13-E14-E15-Xba1.
pCMV-HA-12, expressing an HA-tagged human FUS with 6 preserved RNA motifs in intron 6 and 7, as shown in SEQ ID NO 53. The construction was as followed: Not1-HA-E1 -E2-E3-E4-E5-E6-motif3-motif4-motif1-E7-motif5-motif6-motif2-E8-E9-E10-E11-E12-E13-E14-E15-Xba1.

After 24 h or 48 h of transfection, proteins and RNA were extracted.

### Western Blot

Cells were washed in PBS1× and lysed in Syn-PER Synaptic Protein Extraction (Thermo Scientific, 87793) according to the manufacturer's instructions. Protein extract were dosed by BCA Assay (Interchim, UP95424A, UP95425A). Thereafter proteins were denatured and SDS page were performed with 10 (for cytoplasmic proteins) and 30 μg of protein (for nuclear proteins) on criterion TGX stain free gel 4-20% (Biorad, 5678094). Proteins were blotted on nitrocellulose membrane using semi-dry Transblot Turbo system (BioRad, France) and blocked with 10% non-fat milk during 1 h. Primary antibodies (Rabbit anti-FUS 293 (Bethyl, A-300-293A, 1/2000), Rabbit anti-HA (Cell signaling, 3724S, 1/1000), Mouse anti-MYC (Sigma, M4439, 1/1000)) were incubated overnight at 4° C. in 3% non-fat milk. Washing were proceeded with washing buffer (Tris pH 7.4 1 M, NaCl 5M, Tween 20 100%) and secondary antibody (anti-rabbit HRP (PARIS, BI2407, 1/5000), anti-mouse HRP (Jackson, 115-035-003, 1/5000)) were incubated 1 h 30 at room temperature. After successive washes, proteins were visualized with chemiluminescence using ECL Lumina Forte (Millipore, France) and chemiluminescence detector (Bio-Rad, France). Total proteins were detected with stain free gel capacity (Biorad, 5678094) and used to normalized.

### Antibodies:

Rabbit anti-FUS 293 (Bethyl, A-300-293A, 1/2000)

Rabbit anti-HA (Cell signaling, 3724S, 1/1000)

Mouse anti-MYC (Sigma, M4439, 1/1000)

Anti-rabbit HRP (PARIS, BI2407, 1/5000)

Anti-mouse HRP (Jackson, 115-035-003, 1/5000)

RT-qPCR

Total RNA was extracted from spinal cord and frontal cortex using TRIzol® reagent (Life Technologies). 1 μg of RNA was reverse transcribed with iScript™ reverse transcription (Biorad, 1708841). Quantitative polymerase chain reaction was performed using Sso Advanced Universal SYBR Green Supermix (Bio-Rad) and quantified with Bio-Rad software. Gene expression was normalized by calculating a normalization factor using actin, TBP and pol2 genes according to GeNorm software (Vandesompe et al 2002).

Primer sequences were as follows:

Human ACTIN B:
F-
(SEQ ID NO 60)
GGGCATGGGTCAGAAGGATT,
R-
(SEQ ID NO 61)
TCGATGGGGTACTTCAGGGT
Human GAPDH:
F-
(SEQ ID NO 62)
TTCACCACCATGGAGAAGGC,
R-
(SEQ ID NO 63)
AGAGGGGGCAGAGATGATGA
Human HPRT1:
F-
(SEQ ID NO 64)
TTGCTTTCCTTGGTCAGGCA,
R-
(SEQ ID NO 65)
ATCCAACACTTCGTGGGGTC
FUSi6e7:
F-
(SEQ ID NO 66)
CCGTTGGAAGCTTCATGTCC,
R-
(SEQ ID NO 67)
TATTGAAGCCACCACGGTCAC
FUSi7e8:
F-
(SEQ ID NO 68)
CTGTGAGCACTTACTTGATATTTT,
R-
(SEQ ID NO 69)
GTGATCCTTGGTCCCGA

### LIST OF REFERENCES

1. Murray E. J. (ed.), Methods in Molecular Biology, Vol. 7, Gene Transfer and Expression Protocols, Humana Press (1991)
2. Johnston, Nature, 346: 776-777 (1990)
3. Brash et al., Mol. Cell Biol., 7: 2031-2034 (1987)
4. Smith and Waterman, Adv. Appl. Math., 2:482 (1981)
5. Needleman and Wunsch, J. Mol. Biol., 48:443 (1970)
6. Pearson and Lipman, Proc. Nat. Acad. Sci U.S.A., 85:2444 (1988)
7. Krützfeldt J, Rajewsky N, Braich R, Rajeev K G, Tuschl T, Manoharan M, Stoffel M. Nature. 2005 Dec. 1; 438(7068):685-9. Epub 2005 Oct. 30
8. Jelena Acta Neuropathol 2017
9. Lopez-Erauskin J, Tadokoro T, Baughn M W, et al. ALS/FTD-Linked Mutation in FUS Suppresses Intra-axonal Protein Synthesis and Drives Disease Without Nuclear Loss-of-Function of FUS. Neuron 2018; 100(4): 816-30 e7
10. Scekic-Zahirovic J, Sendscheid O, El Oussini H, et al. Toxic gain of function from mutant FUS protein is crucial to trigger cell autonomous motor neuron loss. EMBO J 2016; 35(10): 1077-97
11. Scekic-Zahirovic J, Oussini H E, Mersmann S, et al. Motor neuron intrinsic and extrinsic mechanisms contribute to the pathogenesis of FUS-associated amyotrophic lateral sclerosis. Acta Neuropathol 2017; 133(6): 887-906.
12. Mitchell J C, 2013 “Overexpression of human wild-type FUS causes progressive motor neuron degeneration in an age- and dose-dependent fashion” Acta Neuropathologica volume 125, pages 273-288(2013 DOI: 10.1007/s00401-012-1043-z.
13. Zhou et al.: “ALS-associated FUS mutations result in compromised FUS alternative splicing and autoregulation. October 2013, Volume 9, Issue 10, e1003895.

1. A nucleic acid encoding human FUS protein comprising a sequence having at least 69% sequence identity with the sequence SEQ ID NO 1 of intron 6 and/or a sequence having at least 60% sequence identity with the sequence SEQ ID NO 2 of intron 7, and/or at least one preserved RNA motif within sequence SEQ ID NO 1 or SEQ ID NO 2 wherein the at least one preserved RNA motif is able to bind human FUS protein said sequences being located on either side of exon 7.

2. The nucleic acid according to claim 1, wherein said sequence having at least 69% sequence identity with the sequence SEQ ID NO. 1 is selected from the group consisting of the sequence SEQ ID NO. 3, SEQ ID NO. 4 and SEQ ID NO. 19.

3. The nucleic acid according to claim 1, wherein said sequence having at least 60% sequence identity with the sequence SEQ ID NO 2 is selected from the group consisting of the sequence SEQ ID NO 5, SEQ ID NO 6, SEQ ID NO 7, SEQ ID NO 8, and SEQ ID NO 20.

4. The nucleic acid according to claim 1, wherein said sequence having a motif within sequence SEQ ID NO 1 able to bind human FUS protein is selected from the group consisting of the sequences SEQ ID NO 54 and SEQ ID NO 55.

5. The nucleic acid according to claim 1, wherein said sequence having a motif within sequence SEQ ID NO 2 able to bind human FUS protein is selected from the group consisting of the sequences SEQ ID NO 56, SEQ ID NO 57, SEQ ID NO 58 and SEQ ID NO 59.

6. The nucleic acid according to claim 1, wherein said sequence is a sequence selected from the group consisting of the sequence SEQ ID NO 9, SEQ ID NO 10, SEQ ID NO 11, SEQ ID NO 12, SEQ ID NO 13, SEQ ID NO 14, SEQ ID NO15, SEQ ID NO 16, SEQ ID NO 17, SEQ ID NO 18, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52 and SEQ ID NO 53.

7. A recombinant vector comprising a nucleic acid according to claim 1.

8. The according to claim 7, characterized in that it is an adenovirus, a plasmid, a YAC (Yeast Artificial Chromosomes) or a BAC (Bacterial Artificial Chromosome).

9. The vector according to claim 7, characterized in that it is an adenovirus vector.

10. A method of treating amyotrophic lateral sclerosis (ALS) comprising administering to a subject in need thereof the nucleic acid of claim 1.

11. A method of treating amyotrophic lateral sclerosis (ALS) comprising administering to a subject in need thereof the vector of claim 7.

12. A pharmaceutical composition for the prevention and/or treatment of amyotrophic lateral sclerosis (ALS), comprising a therapeutically effective amount of the nucleic acid according to claim 1.

13. A host cell comprising the nucleic acid according to claim 1.

13. A pharmaceutical composition for the prevention and/or treatment of amyotrophic lateral sclerosis (ALS), comprising a therapeutically effective amount of the vector according to claim 7.

14. A host cell comprising the vector according to claim 7.
